SEQUENCE LISTING (43 sequences)

SEQ ID NO: 1 - DNA, 119 bp, synthetic construct
  source 1..119: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 2 - DNA, 63 bp, synthetic construct
  source 1..63: mol_type = other DNA
ccctagaaagataatcatattgtgacgtacgttaaagataatcatgcgtaaaattgacgcatg

SEQ ID NO: 3 - DNA, 175 bp, synthetic construct
  source 1..175: mol_type = other DNA
tgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 4 - DNA, 231 bp, synthetic construct
  source 1..231: mol_type = other DNA
agggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 5 - DNA, 218 bp, Pongo abelii
  source 1..218: mol_type = genomic DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 6 - DNA, 100 bp, Mus musculus
  source 1..100: mol_type = genomic DNA
cactgggaggatgttgagtaagatggaaaactactgatgacccttgcagagacagagtattaggacatgtttgaacaggggccgggcgatcagcaggtag

SEQ ID NO: 7 - DNA, 202 bp, Mus musculus
  source 1..202: mol_type = genomic DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 8 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 9 - DNA, 1386 bp, synthetic construct
  source 1..1386: mol_type = other DNA
atgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtga

SEQ ID NO: 10 - DNA, 278 bp, Homo sapiens
  source 1..278: mol_type = genomic DNA
ctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 11 - DNA, 192 bp, synthetic construct
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 12 - DNA, 231 bp, synthetic construct
  source 1..231: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 13 - DNA, 274 bp, synthetic construct
  source 1..274: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgtcatttagaaagagagagcaatatttcaagaatg

SEQ ID NO: 14 - DNA, 35 bp, unidentified
  source 1..35: mol_type = genomic DNA
catgcgtcaattttacgcagactatctttctaggg

SEQ ID NO: 15 - DNA, 894 bp, synthetic construct
  source 1..894: mol_type = other DNA
gaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcg

SEQ ID NO: 16 - DNA, 161 bp, synthetic construct
  source 1..161: mol_type = other DNA
cctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 134 bp, synthetic construct
  source 1..134: mol_type = other DNA
acacagatccacaagctcctgctagcaggtaagtgccgtgtgtggttcccgcgggcctggcctctttacgggttatggcccttgcgtgccttgaattactgacactgacatccactttttctttttctccacag

SEQ ID NO: 18 - DNA, 6491 bp, synthetic construct
  source 1..6491: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcgccaccatgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgaggtaccgatcacatatgcctttaattaaacaactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctgtcgacggaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctatgtacatattatgatatagatacaacgtatgcaatggccaatagccaatattgatttatgctatataaccaatgaataatatggctaatggccaatattgacatgcattgggaggagaccgggacgagacgaaccctgataaatgcttcaataatattatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaataggcgcccttaaggctagctaga

SEQ ID NO: 19 - DNA, 35 bp, unidentified
  source 1..35: mol_type = genomic DNA
caggagtcaattttacgcagactatctttctaggg

SEQ ID NO: 20 - DNA, 471 bp, synthetic construct
  source 1..471: mol_type = other DNA
actaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggag

SEQ ID NO: 21 - DNA, 6623 bp, synthetic construct
  source 1..6623: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgaggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgtcatttagaaagagagagcaatatttcaagaatgcaggagtcaattttacgcagactatctttctagggttaatctagctagccttaagggcgctttcctgtcgacgactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctatgtacatattatgatatagatacaacgtatgcaatggccaatagccaatattgatttatgctatataaccaatgaataatatggctaatggccaatattgacatgcattgggaggagaccgggacgagacgaaccctgataaatgcttcaataatattatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaataggcgcccttaaggctagctagacctgcaggcag

SEQ ID NO: 22 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 23 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 24 - protein, 593 aa, synthetic construct
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 25 - protein, 593 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 26 - protein, 7 aa, synthetic construct
PKKKRKV

SEQ ID NO: 27 - DNA, 21 bp, synthetic construct
  source 1..21: mol_type = other DNA
cccaagaagaagcggaaagtt

SEQ ID NO: 28 - protein, 5 aa, synthetic construct
GGGGS

SEQ ID NO: 29 - DNA, 15 bp, synthetic construct
  source 1..15: mol_type = other DNA
ggcggcggaggcagc

SEQ ID NO: 30 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 31 - DNA, 1815 bp, synthetic construct
  source 1..1815: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 32 - protein, 603 aa, synthetic construct
PKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 33 - DNA, 1809 bp, synthetic construct
  source 1..1809: mol_type = other DNA
cccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 34 - protein, 340 aa, synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 35 - protein, 339 aa, synthetic construct
GKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 36 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 37 - protein, 339 aa, synthetic construct
GKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 38 - protein, 1496 aa, synthetic construct
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 39 - protein, 1495 aa, synthetic construct
SKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 40 - protein, 649 aa, synthetic construct
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 41 - protein, 648 aa, synthetic construct
EEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 42 - protein, 636 aa, synthetic construct
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 43 - protein, 635 aa, synthetic construct
MLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

(19) United States
(12) Patent Application Publication
(10) Pub. No.: US 2025/0381300 A1
(43) Pub. Date: Dec. 18, 2025

(54) AAV PIGGYBAC TRANSPOSON POLYNUCLEOTIDE COMPOSITIONS AND METHODS OF USE THEREFOR

(71) Applicant: Poseida Therapeutics, Inc., San Diego, CA (US)

(72) Inventors: Matthew D. NITZAHN, San Diego, CA (US); Blair B. MADISON, San Diego, CA (US)

(21) Appl. No.: 19/303,242

(22) Filed: Aug. 18, 2025

Related U.S. Application Data
(63) Continuation of application No. PCT/US2024/016679, filed on Feb. 21, 2024.
(60) Provisional application No. 63/486,168, filed on Feb. 21, 2023.

(51) Int. Cl. A61K 48/00; A61K 9/1271; A61K 9/51; A61K 38/44; A61P 3/00; C12N 9/12; C12N 15/86

(52) U.S. Cl. CPC A61K 48/005; A61K 9/1271; A61K 9/5123; A61K 38/44; A61K 48/0083; A61P 3/00; C12N 9/1241; C12N 15/86; C12Y 114/16001; C12N 2750/14143; C12N 2800/22; C12N 2830/50; C12Y 207/07

(57) ABSTRACT

The present disclosure relates to compositions and methods for treating phenylketonuria (PKU). In particular, the present disclosure relates to AAV-piggyBac transposon polynucleotide vectors and LNP compositions comprising a nucleic acid encoding a transposase, and methods of using the compositions for treating PKU.

### RELATED APPLICATIONS

This application claims priority to, and the benefit of, U.S. Provisional Application No. 63/486,168, filed on Feb. 21, 2023. The contents of this application are incorporated herein by reference in its entirety.

### REFERENCE TO AN ELECTRONIC SEQUENCE LISTING

The contents of the electronic sequence listing (POTH-078_001WO_SeqListing_ST26.xml; Size 70,664 bytes in size; and Date of Creation: Feb. 12, 2024) are herein incorporated by reference in its entirety.

### FIELD

The present disclosure relates generally to novel AAV piggyBac transposon polynucleotides comprising a human phenylalanine hydroxylase (PAH) gene, AAV piggyBac vectors comprising said polynucleotides, compositions comprising the AAV piggyBac vectors and lipid nanoparticles (“LNPs”) comprising an mRNA encoding a transposase, methods of preparing these polynucleotides and LNPs, and the use of these AAV piggyBac vectors and LNPs for gene therapy applications, particularly for the treatment of phenylketonuria (PKU).

### BACKGROUND

There has been a long-felt but unmet need in the art for compositions and methods for delivering nucleic acids to cells and for genetically modifying cells in vivo, ex vivo and in vitro. Widely accepted gene delivery and genetic modification techniques, such as the use of viral vectors, including AAVs, can cause acute toxicity and harmful side-effects in patients. The present disclosure provides improved AAV piggyBac transposon polynucleotide compositions comprising a human phenylalanine hydroxylase (PAH) gene, methods for the delivery of said polynucleotide compositions in AAV piggyBac vectors in combination with lipid nanoparticle (LNP) compositions comprising an mRNA encoding a transposase, e.g., a Super PiggyBac Transposase (SPB), to cells, including hepatocytes, in vivo with high efficiency and low toxicity. Thus, the compositions and methods of the present disclosure have applicability for gene therapy therapeutics for treating phenylketonuria (PKU).

The present disclosure provides for AAV piggyBac transposon polynucleotides comprising a transgene encoding a codon optimized and modified human phenylalanine hydroxylase (PAH) gene, AAV piggyBac vector compositions comprising the polynucleotides, and methods for using the AAV piggyBac vector compositions in combination with a LNP composition comprising at least one mRNA encoding a transposase, e.g., SPB, for the treatment of phenylketonuria (PKU). The compositions and methods are described in further detail herein.

### SUMMARY OF THE INVENTION

The disclosure provides an adeno-associated virus (AAV) piggyBac transposon polynucleotide comprising in the 5′ to 3′ direction: a) a first AAV ITR sequence; b) a first piggyBac ITR sequence; c) a first piggyBac 3′ UTR; d) a first insulator sequence; e) at least one promoter sequence; f) at least one intron sequence; g) at least one transgene sequence encoding human phenylalanine hydroxylase (PAH); h) a polyA sequence; i) a second insulator sequence; j) a first piggyBac 5′ UTR sequence; k) a second piggyBac ITR sequence; l) at least one DNA spacer sequence; and m) a second AAV ITR sequence.

The disclosure provides an adeno-associated virus (AAV) piggyBac transposon polynucleotide comprising in the 5′ to 3′ direction: a) a first AAV ITR sequence comprising the nucleic acid sequence of SEQ ID NO: 1; b) a first piggyBac ITR sequence comprising the nucleic acid sequence of SEQ ID NO: 2; c) a first piggyBac 3′ UTR sequence comprising the nucleic acid sequence of SEQ ID NO: 3; d) a first insulator sequence comprising the nucleic acid sequence of SEQ ID NO: 4; e) a promoter sequence comprising the nucleic acid sequence of SEQ ID NO: 7; f) a synthetic intron sequence comprising the nucleic acid sequence of SEQ ID NO: 17; g) a transgene sequence encoding a codon optimized and modified human PAH gene comprising the nucleic acid sequence of SEQ ID NO: 9; h) a polyA sequence comprising the nucleic acid sequence of SEQ ID NO: 11; i) a second insulator sequence comprising the nucleic acid sequence of SEQ ID NO: 12; j) a piggyBac 5′UTR sequence comprising the nucleic acid sequence of SEQ ID NO: 13; k) a second piggyBac ITR sequence comprising the nucleic acid sequence of SEQ ID NO: 14; l) a DNA spacer sequence comprising the nucleic acid sequence of SEQ ID NO: 15; and m) a second AAV ITR sequence comprising the nucleic acid sequence of SEQ ID NO: 16.

In some aspects, the AAV piggyBac transposon polynucleotide comprises the nucleic acid sequence of SEQ ID NO: 18.

The disclosure provides an adeno-associated virus (AAV) piggyBac transposon polynucleotide comprising in the 5′ to 3′ direction: a) a first AAV ITR sequence; b) a first piggyBac ITR sequence; c) a first piggyBac 3′ UTR; d) a first insulator sequence; c) at least one enhancer sequence; f) at least one promoter sequence; g) at least one transgene sequence encoding human phenylalanine hydroxylase (PAH); h) a first 3′ UTR; i) a polyA sequence; j) a second insulator sequence; k) a first piggyBac 5 UTR; l) a second piggyBac ITR sequence comprising a 35TCC mutation; m) at least one DNA spacer sequence; and n) a second AAV ITR sequence.

The disclosure provides an adeno-associated virus (AAV) piggyBac transposon polynucleotide comprising in the 5′ to 3′ direction: a) a first AAV ITR sequence; b) a first piggyBac ITR sequence; c) a first piggyBac 3′ UTR; d) a first insulator sequence; e) a 3×hSERPINA1 enhancer; f) a TTR enhancer; g) a TTRm promoter sequence; h) a MVM intron; i) a transgene sequence comprising a nucleic acid sequence encoding a codon optimized and modified human PAH gene; j) a AES-mtRNR 3′ UTR; k) a polyA sequence; l) a second insulator sequence; m) a first piggyBac 5′ UTR; n) a second piggyBac ITR sequence comprising a 35TCC mutation; o) a DNA spacer sequence; and p) a second AAV ITR sequence.

The disclosure provides an adeno-associated virus (AAV) piggyBac transposon polynucleotide comprising in the 5′ to 3′ direction: a) a first AAV ITR sequence comprising the nucleic acid sequence of SEQ ID NO: 1; b) a first piggyBac ITR sequence comprising the nucleic acid sequence of SEQ ID NO: 2: c) a first piggyBac 3′ UTR sequence comprising the nucleic acid sequence of SEQ ID NO: 3; d) a first insulator sequence comprising the nucleic acid sequence of SEQ ID NO: 4; e) a first enhancer sequence comprising the nucleic acid sequence of SEQ ID NO: 5; f) a second enhancer sequence comprising the nucleic acid sequence of SEQ ID NO: 6; g) a promoter sequence comprising the nucleic acid sequence of SEQ ID NO: 7; h) an intron sequence comprising the nucleic acid sequence of SEQ ID NO: 8; i) a transgene sequence encoding a codon optimized and modified human PAH gene comprising the nucleic acid sequence of SEQ ID NO: 9; j) an AES-mtRNR 3′ UTR comprising the nucleic acid of SEQ ID NO. 10; k) a polyA sequence comprising the nucleic acid sequence of SEQ ID NO: 11; l) a second insulator sequence comprising the nucleic acid sequence of SEQ ID NO: 12; m) a piggyBac 5′UTR sequence comprising the nucleic acid sequence of SEQ ID NO: 13; n) a second piggyBac ITR sequence comprising a 35TCC mutation, comprising the nucleic acid sequence of SEQ ID NO: 19; o) a DNA spacer sequence comprising the nucleic acid sequence of SEQ ID NO: 15; and p) a second AAV ITR sequence comprising the nucleic acid sequence of SEQ ID NO: 16.

In some aspects, the AAV piggyBac transposon polynucleotide comprises the nucleic acid sequence of SEQ ID NO: 21.

The disclosure provides a vector comprising any one of the AAV piggyBac transposon polynucleotide of the disclosure. In some aspects, the vector is an AAV viral vector. In some aspects, the AAV viral vector is an AAV1, AAV2, AAV3, AAV4, AAV5, AAV6, AAV7, AAV8, AAV9, AAV10 or AAV11 viral vector. In some aspects, the AAV viral vector is an AAV8 or AAV9 viral vector.

The disclosure provides a pharmaceutical composition comprising any one of the vectors of the disclosure.

The disclosure provides a composition comprising: 1) any one of the vectors of the disclosure; and 2) at least one LNP composition comprising at least one mRNA molecule encoding a transposase. In some aspects, the mRNA molecule further comprises a 5′-CAP. In some aspects, the transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TeBuster transposase.

In some aspects, the at least one LNP composition comprises: about 54% of ssPalmO-Ph-P4C2 by moles, about 35% of cholesterol by moles, about 10% of DOPC by moles, and about 1% of DMG-PEG2000 by moles.

The disclosure provides a pharmaceutical composition comprising any one of the compositions of the disclosure.

The disclosure provides a method of treating phenylketonuria (PKU) in a subject in need thereof comprising administering to the subject at least one therapeutically effective dose of a composition comprising: a) the polynucleotide, vector or pharmaceutical composition of any one of the preceding claims; and b) at least one LNP composition comprising at least one mRNA molecule encoding a transposase.

In some aspects, the at least one LNP composition comprises: about 54% of ssPalmO-Ph-P4C2 by moles, about 35% of cholesterol by moles, about 10% of DOPC by moles, and about 1% of DMG-PEG2000 by moles. In some aspects, the mRNA molecule further comprises a 5′-CAP. In some aspects, the transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase. In some aspects, the transposase is a Super piggyBac™ (SPB) transposase.

### BRIEF DESCRIPTION OF THE DRAWINGS

The above and further features will be more clearly appreciated from the following detailed description when taken in conjunction with the accompanying drawings.

FIG. 1 is a schematic diagram of an exemplary AAV piggyBac transposon polynucleotide comprising a codon optimized and modified human phenylalanine hydroxylase (PAH) gene of the present disclosure.

FIG. 2 is a schematic diagram of a second exemplary AAV piggyBac transposon polynucleotide comprising a codon optimized and modified human PAH gene of the present disclosure.

FIG. 3A is a digital image of a Western Blot of HA-PAH transgene expression levels in wild type mice treated i.v. with vehicle, a low and high dose of an AAV piggyBac transposon polynucleotide and an LNP composition comprising: an mRNA encoding SPB or an mRNA encoding a catalytically inactive SPB, or an mRNA encoding SPB combination via hydrodynamic delivery (HDD).

FIG. 3B is a graph illustrating the amount of HA-tagged PAH expression/mg protein for samples from vehicle, treated and HDD administered mice from FIG. 3A.

FIG. 4 is a graph showing the relative percentage of hepatocytes expressing human PAH in treated mice administered a low dose or high dose of an AAV piggyBac transposon vector and an LNP composition comprising an mRNA encoding SPB or an mRNA encoding a catalytically inactive SPB, or an mRNA encoding SPB via hydrodynamic delivery (HDD).

FIG. 5A is a graph showing the episomal and integrated viral copy number (VCN) in hepatocytes isolated from mice treated with vehicle, a low dose of an AAV piggyBac transposon vector alone, a low dose of AAV piggyBac transposon vector and an LNP composition comprising an mRNA encoding SPB, a high dose of an AAV piggyBac transposon vector alone, a high dose of AAV piggyBac transposon vector and an LNP composition comprising an mRNA encoding a catalytically inactive SPB and a high dose of AAV piggyBac transposon vector and an LNP composition comprising an mRNA encoding SPB.

FIG. 5B is an expanded graph showing the only integrated VCN for samples shown in FIG. 5A.

FIG. 6A shows a graph of serum Phe levels (AM) in male BALB PAH^{enu} mice treated with vehicle, an AAV piggyBac transposon vector (Construct #1) and an LNP composition comprising an mRNA encoding SPB, an AAV piggyBac transposon vector and an LNP composition comprising an mRNA encoding a catalytically inactive SPB or control wild type mice at Days 0, 7, 14 and 28 post administration.

FIG. 6B shows a graph of serum Phe levels (μM) in female BALB PAH^{enu} mice treated with vehicle, an AAV piggyBac transposon vector (Construct #1) and an LNP composition comprising an mRNA encoding SPB, an AAV piggyBac transposon vector and an LNP composition comprising an mRNA encoding a catalytically inactive SPB or control wild type mice at Days 0, 7, 14 and 28 post administration.

FIG. 7A shows a graph of serum Phe levels (μM) in male BALB PAH^{enu} mice treated with vehicle, an AAV piggyBac transposon vector (Construct #2) and an LNP composition comprising an mRNA encoding SPB, an AAV piggyBac transposon vector and an LNP composition comprising an mRNA encoding a catalytically inactive SPB, or control wild type mice at Days 0, 7, 14 and 28 post administration.

FIG. 7B shows a graph of serum Phe levels (μM) in female BALB PAH^{enu} mice treated with vehicle, an AAV piggyBac transposon vector (Construct #2) and an LNP composition comprising an mRNA encoding SPB, an AAV piggyBac transposon vector and an LNP composition comprising an mRNA encoding a catalytically inactive SPB, or control wild type mice at Days 0, 7, 14 and 28 post administration.

FIG. 8A is a graph illustrating the amount of HA-tagged PAH expression/mg protein for samples from wild type juvenile mice treated i.v. with vehicle, AAV piggyBac transposon polynucleotide and an LNP composition comprising an mRNA encoding a catalytically inactive SPB, or AAV piggyBac transposon polynucleotide and an LNP composition comprising an mRNA encoding SPB.

FIG. 8B is a graph showing the episomal and integrated viral copy number (VCN) in hepatocytes isolated from mice treated with vehicle, AAV piggyBac transposon vector and an LNP composition comprising an mRNA encoding a catalytically inactive SPB, and AAV piggyBac transposon vector and an LNP composition comprising an mRNA encoding SPB.

### DETAILED DESCRIPTION

### Compositions of the Present Disclosure—AAV piggyBac Transposon Polynucleotides

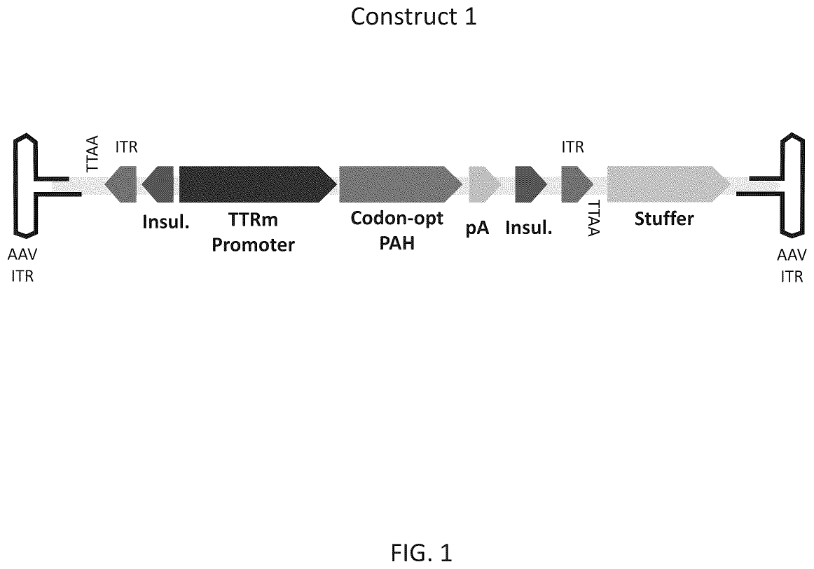

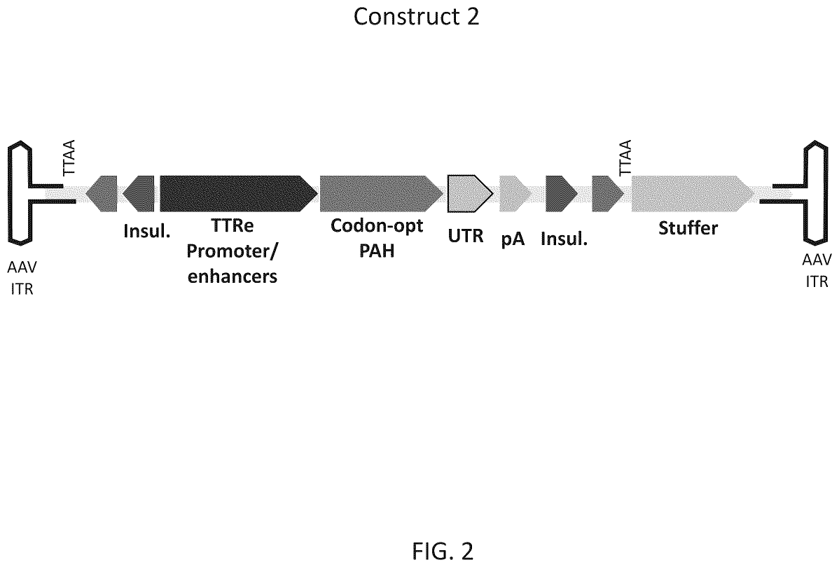

The present disclosure provides an AAV piggyBac transposon polynucleotide comprising a human PAH gene. In some embodiments, the nucleotide sequence of the PAH gene is codon optimized to improve expression of the encoded human PAH enzyme. In some embodiments, the nucleotide sequence of the human PAH gene is modified to remove any internal TTAA sites; any undesired restriction enzyme recognition sites; and any putative cryptic splice sites for cloning the modified sequences into AAV piggyBac transposon vectors. In some embodiments, the nucleotide sequence of the human PAH gene is codon optimized and further modified as described above. Two exemplary AAV piggyBac transposon polynucleotides comprising a codon optimized and modified PAH gene are shown schematically in FIG. 1 and FIG. 2, respectively.

In certain aspects of the present disclosure, the AAV piggyBac transposon polynucleotide comprises in the 5′ to 3′ direction: a) a first AAV ITR sequence; b) a first piggyBac ITR sequence; c) a piggyBac 3′ UTR sequence; d) a first insulator sequence; c) a TTRm promoter sequence; f) a synthetic intron sequence: g) a transgene sequence comprising a nucleic acid sequence encoding a human PAH gene; h) a polyA sequence; i) a second insulator sequence; j) a piggyBac 5′ UTR sequence; k) a second piggyBac ITR; l) a DNA spacer sequence; and m) a second AAV ITR sequence.

In one embodiment, the first AAV ITR sequence comprises the nucleic acid of SEQ ID NO. 1. In one embodiment, the first piggyBac ITR sequence comprises the nucleic acid of SEQ ID NO. 2. In one embodiment, the first piggyBac 3′ UTR sequence comprises the nucleic acid of SEQ ID NO. 3. In one embodiment, the first insulator sequence comprises the nucleic acid of SEQ ID NO. 4. In one embodiment, the TTRm promoter sequence comprises the nucleic acid of SEQ ID NO. 7. In one embodiment, the synthetic intron sequence comprises the nucleic acid of SEQ ID NO. 17. In one embodiment, the transgene PAH nucleotide sequence further comprises a hemagglutinin (HA) tag. In one embodiment, the transgene PAH nucleotide sequence is codon optimized. In one embodiment, the transgene PAH nucleotide sequence is modified. In one embodiment, the transgene PAH nucleotide sequence comprises a hemagglutinin tag, is codon optimized and is further modified as disclosed herein. In one embodiment, the nucleic acid sequence encoding the human PAH gene sequence comprises the nucleic acid of SEQ ID NO. 9. In one embodiment, the polyA sequence comprises the nucleic acid of SEQ ID NO. 11. In one embodiment, the second insulator sequence comprises the nucleic acid of SEQ ID NO. 12. In one embodiment, the piggyBac 5′ UTR sequence comprises the nucleic acid of SEQ ID NO. 13. In one embodiment, the second piggyBac ITR comprises the nucleic acid of SEQ ID NO. 14. In one embodiment, the DNA spacer sequence comprises the nucleic acid of SEQ ID NO. 15. In one embodiment, the second AAV ITR sequence comprises the nucleic acid of SEQ ID NO. 16.

In certain aspects of the present disclosure, the first AAV piggyBac transposon polynucleotide comprises in the 5′ to 3′ direction: a) a first AAV ITR sequence comprising the nucleic acid of SEQ ID NO. 1; b) a first piggyBac ITR sequence comprising the nucleic acid of SEQ ID NO. 2; c) a piggyBac 3′ UTR comprising the nucleic acid of SEQ ID NO. 3; d) a first insulator sequence comprising the nucleic acid of SEQ ID NO. 4; e) a TTRm promoter sequence comprising the nucleic acid of SEQ ID NO. 7; f) a synthetic intron sequence comprising the nucleic acid of SEQ ID NO. 17; g) a transgene sequence comprising a nucleic acid sequence encoding a hemagglutinin (HA)-tagged, codon optimized and modified human PAH gene comprises the nucleic acid of SEQ ID NO. 9; h) a polyA sequence comprising the nucleic acid of SEQ ID NO. 11; i) a second insulator sequence comprising the nucleic acid of SEQ ID NO. 12; j) a piggyBac 5′ UTR sequence comprising the nucleic acid of SEQ ID NO. 13; k) a second piggyBac ITR comprising the nucleic acid of SEQ ID NO. 14; l) a DNA spacer sequence comprising the nucleic acid of SEQ ID NO. 15; and m) a second AAV ITR sequence comprising the nucleic acid of SEQ ID NO. 16.

In certain aspects, the AAV piggyBac transposon polynucleotide comprises in the 5′ to 3′ direction: a) a first AAV ITR sequence (SEQ ID NO. 1); b) a first piggyBac ITR sequence (SEQ ID NO. 2); c) a piggyBac 3′ UTR sequence (SEQ ID NO. 3): d) a first insulator sequence (SEQ ID NO. 4); e) a TTRm promoter region comprising a 3×hSERPINA1 enhancer (SEQ ID NO. 5), a TTR enhancer (SEQ ID NO. 6), a TTRm promoter sequence (SEQ ID NO. 7), and a MVM intron (SEQ ID NO. 8); f) a transgene sequence comprising a nucleic acid sequence encoding a hemagglutinin (HA)-tagged, codon optimized and modified human PAH gene (SEQ ID NO. 9) followed by a AES-mtRNR 3′ UTR (SEQ ID NO. 10); g) a polyA sequence (SEQ ID NO. 11); h) a second insulator sequence (SEQ ID NO. 12); i) a piggyBac 5′ UTR sequence (SEQ ID NO. 13) a second piggyBac ITR sequence comprising a 35TCC mutation (SEQ ID NO. 19); j) a DNA spacer sequence (SEQ ID NO. 20); and k) a second AAV ITR sequence (SEQ ID NO. 16).

### Compositions of the Present Disclosure—Lipid Nanoparticles

The present disclosure provides a composition comprising at least one lipid nanoparticle comprising at least one cationic lipid and at least one nucleic acid molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

Accordingly, the present disclosure provides compositions comprising at least one lipid nanoparticle, wherein the at least one lipid nanoparticle comprises at least one cationic lipid, at least one nucleic acid molecule, at least one structural lipid, at least one phospholipid and at least one PEGylated lipid.

### Bioreducible Ionizable Cationic Lipids

In some aspects, a cationic lipid can be a bioreducible ionizable cationic lipid.

Accordingly, the present disclosure provides compositions comprising at least one lipid nanoparticle, wherein the at least one lipid nanoparticle comprises at least one bioreducible ionizable cationic lipid.

As used herein, the term “bioreducible ionizable cationic lipid” is used in its broadest sense to refer to a cationic lipid comprising: at least one tertiary amine, at least one disulfide group, at least one group comprising a bond that is susceptible to cleavage by thioesterification, and further comprising at least two saturated or unsaturated hydrocarbon chains. Exemplary bioreducible ionizable cationic lipids include, but are not limited to, those described in Akita et al., (2020) Biol. Phar. Bull. 43:1617-1625, the contents of which is incorporated herein by reference in their entirety.

Additional exemplary bioreducible ionizable cationic lipids and methods of preparing such lipids useful in the methods of the present disclosure include those disclosed in International Patent Application No. PCT/JP2016/052690, published as WO/2016/121942 and International Patent Application No. PCT/JP2019/012302, published as WO/2019/188867, the contents of each of which are incorporated herein by reference in their entirety.

Accordingly, the present disclosure provides compositions comprising at least one lipid nanoparticle, wherein the at least one lipid nanoparticle comprises any one of the bioreducible ionizable cationic lipids put forth in WO/2016/121942 and WO/2019/188867.

Accordingly, the present disclosure provides compositions comprising at least one lipid nanoparticle, wherein the at least one lipid nanoparticle comprises at least one bioreducible ionizable cationic lipid, at least one nucleic acid molecule, at least one structural lipid, at least one phospholipid and at least one PEGylated lipid.

In some aspects, the bioreducible ionizable cationic lipid can be ssPalmO-Ph-P4C2, having the following structure:

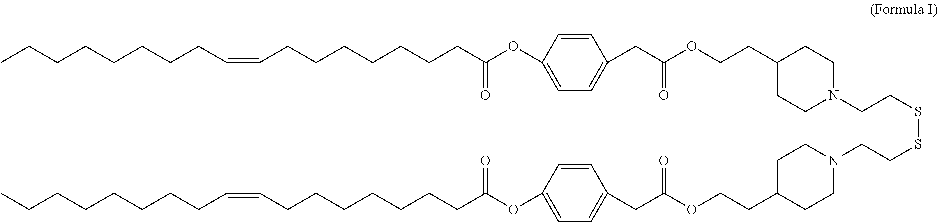 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

See Akita et al., (2020) Biol. Phar. Bull. 43:1617-1625, the contents of which are incorporated by reference in their entirety.

Accordingly, the present disclosure provides compositions comprising at least one lipid nanoparticle comprising at least one bioreducible ionizable cationic lipid, wherein the at least one bioreducible ionizable cationic lipid comprises ssPalmO-Ph-P4C2.

As would be appreciated by the skilled artisan, ssPalmO-Ph-P4C2 can also be referred to as Coatsome® SS-OP, ssPalmO-Phe-P4C2, ssPalmO-Phenyl-P4C2, ssPalmO-Phe and ssPalmO-Ph. Accordingly, ssPalmO-Ph-P4C2, Coatsome® SS-OP, ssPalmO-Phe-P4C2, ssPalmO-Phenyl-P4C2, ssPalmO-Phe and ssPalmO-Ph are used interchangeably herein to refer to the bioreducible ionizable cationic lipid with the chemical structure put forth in Formula I.

As described herein, the LNP compositions of the present disclosure that comprise at least one bioreducible ionizable cationic lipid advantageously exhibit significantly reduced toxicity in animals as compared to LNP compositions comprising non-bioreducible ionizable cationic lipids. In particular, administration the LNP compositions of the present disclosure surprisingly does not result in any body weight loss. In some aspects, certain LNP compositions of the present disclosure are so non-toxic that animals administered the LNPs actually gain body weight, even when administered amounts of LNPs that exceed the lethal dose of LNP compositions comprising non-bioreducible ionizable cationic lipids.

### LNP Components

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one bioreducible ionizable cationic lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one bioreducible ionizable cationic lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5%, or about 60%, or about 62.5%, or about 65%, or about 67.5%, or about 70% of at least one phospholipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5%, or at least about 60%, or at least about 62.5%, or at least about 65%, or at least about 67.5%, or at least about 70% of at least one phospholipid by moles.

In some aspects, an LNP of the present disclosure can comprise about 0.25%, or about 0.5%, or about 0.75%, or about 1.0%, or about 1.25%, or about 1.5%, or about 1.75%, or about 2.0%, or at least about or about 2.5%, or about 5% of at least one PEGylated lipid by moles.

In some aspects, an LNP of the present disclosure can comprise at least about 0.25%, or at least about 0.5%, or at least about 0.75%, or at least about 1.0%, or at least about 1.25%, or at least about 1.5%, or at least about 1.75%, or at least about 2.0%, or at least about 2.5%, or at least about 5% of at least one PEGylated lipid by moles.

### Structural Lipids

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

### Phospholipid

As used herein, the term “phospholipid” is used in its broadest sent to refer to any amphiphilic molecule that comprises a polar (hydrophilic) headgroup comprising phosphate and two hydrophobic fatty acid chains.

In some aspects of the lipid nanoparticles of the present disclosure, a phospholipid can comprise dioleoylphosphatidylethanolamine (DOPE).

In some aspects of the lipid nanoparticles of the present disclosure, a phospholipid can comprise DOPC (1,2-Dioleoyl-sn-glycero-3-phosphocholine).

In some aspects of the lipid nanoparticles of the present disclosure, a phospholipid can comprise DSPC (1,2-Distearoyl-sn-glycero-3-phosphocholine).

In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3[Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3[Phospho-rac-(1-glycerol) (Sodium Salt)), DLPG-NH4 (1,2-Dilauroyl-sn-glycero-3[Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3[Phospho-rac-(1-glycerol) (Sodium Salt)), DMPG-NH4 (1,2-Dimyristoyl-sn-glycero-3[Phospho-rac-(1-glycerol) (Ammonium Salt)), DMPG-NH4/NA (1,2-Dimyristoyl-sn-glycero-3[Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3[Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3[Phospho-rac-(1-glycerol) (Sodium Salt)), DPPG-NH4 (1,2-Dipalmitoyl-sn-glycero-3[Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3[Phospho-rac-(1-glycerol) (Sodium Salt)), DSPG-NH4 (1,2-Distearoyl-sn-glycero-3[Phospho-rac-(1-glycerol) (Ammonium Salt)), DSPS-NA (1,2-Distearoyl-sn-glycero-3-phosphoserine (Sodium Salt)), EPC (Egg-PC), HEPC (Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3[Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

### PEGylated Lipid

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypoly ethylene glycol-2000, hereafter referred to as DMG-PEG2000.

### Nucleic Acids

In some aspects, a lipid nanoparticle can comprise at least one nucleic acid molecule. In some aspects, a lipid nanoparticle can comprise a plurality of nucleic acid molecules. In some aspects, the at least one nucleic acid molecule or the plurality of nucleic acid molecules can be formulated in a lipid nanoparticle.

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art.

In some aspects, a lipid nanoparticle can comprise lipid and nucleic acid at a specified ratio (weight/weight).

In some aspects, a lipid nanoparticle comprising at least one nucleic acid molecule can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid:nucleic acid, weight/weight.

In some aspects, a lipid nanoparticle can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, or about 200:1, lipid:nucleic acid, weight/weight.

In some aspects, a lipid nanoparticle can comprise lipid and nucleic acid at a ratio of about 10:1, or about 17.5:1, or about 25:1, lipid:nucleic acid, weight/weight.

In some aspects, a nucleic acid molecule can be an RNA molecule. Thus, in some aspects, a lipid nanoparticle can comprise at least one RNA molecule. In some aspects, an RNA molecule can be an mRNA molecule. In some aspects, an mRNA molecule can comprise a 5′-CAP.

In some aspects, an mRNA molecule can be capped using any method and/or capping moiety known in the art. An mRNA molecule can be capped with m7G(5′)ppp(5′)G moiety. A m7G(5′)ppp(5′)G moiety is also referred to herein as a “CapO”. An mRNA molecule can be capped with a CleanCap® moiety. A CleanCap® moiety can comprise a m7G(5′)ppp(5′)(2′OMeA) (CleanCap® AG) moiety. A CleanCap® moiety can comprise a m7G(5′)ppp(5′)(2′OMeG) (CleanCap® GG) moiety. An mRNA molecule can be capped with an anti-reverse cap analog (ARCA®) moiety. An ARCA® moiety can comprise a m7(3′-O-methyl)G(5′)ppp(5′)G moiety. An mRNA molecule can be capped with a CleanCap® 3′OMe moiety (CleanCap®+ARCA®).

In some aspects, an mRNA molecule can comprise at least one modified nucleic acid.

Modified nucleic acids can include, but are not limited to, 5-methoxy uridine (5moU), N₁-methyl pseudouridine (me¹ψ), pseudouridine (Y), 5-methylcytidine (5-MeC).

In some aspects, a nucleic acid molecule can be a DNA molecule. Thus, in some aspects, a lipid nanoparticle can comprise at least one DNA molecule. In some aspects, a DNA molecule can be a circular DNA molecule, such as, but not limited to, a DNA plasmid. In some aspects, a lipid nanoparticle can comprise a DNA plasmid. In some aspects, a DNA molecule can be a linearized DNA molecule, such as, but not limited to, a linearized DNA plasmid. In some aspect, a DNA molecule can be a DoggyBone DNA molecule. In some aspects, a DNA molecule can be a DNA nanoplasmid.

A DNA plasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

### LNP Compositions

In some aspects, a lipid nanoparticle can comprise at least one nucleic acid molecule, at least one bioreducible ionizable cationic lipid, and at least one structural lipid. In some aspects, a lipid nanoparticle can comprise at least one nucleic acid molecule, at least one bioreducible ionizable cationic lipid, and at least one PEGylated lipid. In some aspects, the at least one bioreducible ionizable cationic lipid can be ssPalmO-Ph-P4C2.

In some aspects, the at least one structural lipid can be a mixture of two structural lipids. In some aspects, the at least one PEGylated lipid can be a mixture of two PEGylated lipids.

In some aspects, a lipid nanoparticle can comprise at least one nucleic acid molecule, at least one bioreducible ionizable cationic lipid, at least one structural lipid, at least one PEGylated lipid or any combination thereof. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, and at least one PEGylated lipid. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid, at least one PEGylated lipid or any combination thereof. In some aspects, the at least one bioreducible ionizable cationic lipid can be ssPalmO-Ph-P4C2.

In some aspects, a lipid nanoparticle can comprise at least one nucleic acid molecule, at least one bioreducible ionizable cationic lipid, at least one structural lipid, at least one phospholipid and at least one PEGylated lipid. In some aspects, the at least one bioreducible ionizable cationic lipid can be ssPalmO-Ph-P4C2.

In some aspects, the nucleic acid molecule is a RNA molecule. Thus, in some aspects, a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a lipid nanoparticle comprising at least one nucleic acid molecule can comprise between about 44% to 64% of ssPalmO-Ph-P4C2 by moles; between about 25% to 45% of cholesterol by moles, between about 0.1% to 20% of DOPC by moles, and between about 0.1% to 11% of DMG-PEG2000 by moles, wherein the at least one lipid nanoparticle comprises at least one nucleic acid molecule, wherein the at least one nucleic acid molecule comprises at least one RNA molecule. In some aspects, a lipid nanoparticle comprising at least one nucleic acid molecule can comprise between about 49% to 59% of ssPalmO-Ph-P4C2 by moles; between about 30% to 40% of cholesterol by moles, between about 5% to 15% of DOPC by moles, and between about 0.5% to 6% of DMG-PEG2000 by moles, wherein the at least one lipid nanoparticle comprises at least one nucleic acid molecule, wherein the at least one nucleic acid molecule comprises at least one RNA molecule. In some aspects, the mRNA molecule further comprises a 5′-CAP. In some aspects, the ratio of lipid to nucleic acid in the at least one nanoparticle can be about 75:1 to about 100:1 (w/w).

In some aspects, the nucleic acid molecule is a DNA molecule. Thus, the present disclosure provides a lipid nanoparticle comprising at least one nucleic acid molecule can comprise about 54% ssPalmO-Ph-P4C2 by moles, about 35% cholesterol by moles, about 10% of DOPC by moles, and about 1% DMG-PEG2000 by moles, wherein the lipid nanoparticle further comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA (see WO/2020/154645). In some aspects, the ratio of lipid to nucleic acid in the nanoparticle can be about 100:1 (w/w).

In some aspects, a lipid nanoparticle comprising at least one nucleic acid molecule can comprise between about 44% to 64% of ssPalmO-Ph-P4C2 by moles; between about 25% to 45% of cholesterol by moles, between about 0.1% to 20% of DOPC by moles, and between about 0.1% to 11% of DMG-PEG2000 by moles, wherein the at least one lipid nanoparticle comprises at least one nucleic acid molecule, wherein the at least one nucleic acid molecule comprises at least one DNA molecule. In some aspects, a lipid nanoparticle comprising at least one nucleic acid molecule can comprise between about 49% to 59% of ssPalmO-Ph-P4C2 by moles; between about 30% to 40% of cholesterol by moles, between about 5% to 15% of DOPC by moles, and between about 0.5% to 6% of DMG-PEG2000 by moles, wherein the at least one lipid nanoparticle comprises at least one nucleic acid molecule, wherein the at least one nucleic acid molecule comprises at least one DNA molecule. In some aspects, the at least one DNA molecule can be a DoggyBone DNA molecule. In some aspects, the at least one DNA molecule can be a DNA nanoplasmid. In some aspects, the at least one DNA molecule can be a covalently closed ended DNA. In some aspects, the ratio of lipid to nucleic acid in the at least one nanoparticle can be about 75:1 to about 100:1 (w/w).

Exemplary LNP compositions and methods of preparing such LNP compositions useful in the methods of the present disclosure include those disclosed in International Patent Application No. PCT/US2022/017570, published as WO/2022/182792, the contents of which is incorporated herein by reference in its entirety.

Accordingly, the present disclosure provides LNP compositions comprising any one of the LNP compositions put forth in WO/2022/182792.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present disclosure by combining a miscible solvent phase comprising the lipid components of the nanoparticle and an aqueous phase comprising the lipid nanoparticle cargo (e.g. nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr®) Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a T-mixer, wherein the T-mixer mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

In some aspects, the lipid nanoparticles of the present disclosure can be produced using a T-mixer, wherein the T-mixer mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

Compositions of the Present Disclosure-Adeno-Associated Virus (AAV) piggyBac Transposon Polynucleotides

The present disclosure provides compositions comprising adeno-associated virus (AAV) piggyBac transposon polynucleotides.

In some aspects an AAV piggyBac transposon polynucleotide can comprise at least one AAV inverted terminal repeat (ITR) sequence. In some aspects, an AAV piggyBac transposon polynucleotide can comprise at least one piggyBac ITR sequence. In some aspects, an AAV piggyBac transposon polynucleotide can comprise at least one insulator sequence. In some aspects an AAV piggyBac transposon polynucleotide can comprise at least one enhancer sequence. In some aspects an AAV piggyBac transposon polynucleotide can comprise at least one promoter sequence. In some aspects, an AAV piggyBac transposon polynucleotide can comprise at least one transgene sequence. In some aspects, an AAV piggyBac transposon polynucleotide can comprise at least one poly A sequence. In some aspects, an AAV piggyBac transposon polynucleotide can comprise at least one DNA spacer sequence. In some aspects, an AAV piggyBac transposon polynucleotide can comprise at least one piggyBac 3′UTR sequence. In some aspects, an AAV piggyBac transposon polynucleotide can comprise at least one piggyBac 5′UTR sequence. In some aspects, an AAV piggyBac transposon polynucleotide can comprise at least one 3′UTR sequence. In some aspects, an AAV piggyBac transposon polynucleotide can comprise at least one intron sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, and at least one polyA sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, and at least one polyA sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence, wherein between the first insulator sequence and the second insulator sequence there is any combination of at least one promoter sequence, at least one transgene sequence, and at least one polyA sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, and followed by a second AAV ITR sequence.

In some aspects, an AAV piggyBac transposon polynucleotide can comprise in between a second piggyBac ITR sequence and a second AAV ITR sequence, at least one DNA spacer sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second AAV ITR sequence.

In a non-limiting example of the preceding AAV piggyBac transposon polynucleotides, the at least one transgene sequence can comprise a nucleic acid sequence that encodes for a human phenylalanine hydroxylase (hPAH) polypeptide. In certain aspects, the nucleotide sequence encoding hPAH is codon optimized. In certain aspects, the codon optimized PAH gene is further modified to i) remove any internal TTAA sites; ii) any undesired restriction enzyme recognition sites; and iii) putative cryptic splice sites for cloning the modified sequences into AAV piggyBac transposon vectors. This non-limiting example of an AAV piggyBac transposon polynucleotide is shown in FIG. 1.

An AAV piggyBac transposon polynucleotide can comprise a first AAV ITR sequence, a first piggyBac ITR sequence, a first insulator sequence, at least one enhancer sequence, at least one promoter sequence, a first transgene sequence, at least one 3′ UTR, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one enhancer sequence, at least one promoter sequence, a first transgene sequence, at least one 3′ UTR, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one enhancer sequence, followed by at least one promoter sequence, followed by a first transgene sequence, followed by at least one 3′ UTR, followed by a polyA sequence, followed by a second insulator sequence, followed by a second piggyBac ITR sequence, and followed by a second AAV ITR sequence.

In some aspects, an AAV piggyBac transposon polynucleotide can comprise in between a second piggyBac ITR sequence and a second AAV ITR sequence, at least one DNA spacer sequence.

An AAV piggyBac transposon polynucleotide can comprise a first AAV ITR sequence, a first piggyBac ITR sequence, a first insulator sequence, at least one enhancer sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one enhancer sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one enhancer sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second AAV ITR sequence.

In some aspects, an AAV piggyBac transposon polynucleotide can comprise more than one enhancer sequence. In some aspects wherein the AAV piggyBac transposon polynucleotide comprises more than one enhancer sequence, the enhancer sequences can be the same or the enhancer sequences can be different.

An AAV piggyBac transposon polynucleotide can comprise a first AAV ITR sequence, a first piggyBac ITR sequence, a first insulator sequence, a first enhancer sequence, a second enhancer sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, a first enhancer sequence, a second enhancer sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise a first AAV ITR sequence, followed by a first piggyBac ITR sequence, followed by a first insulator sequence, followed by a first enhancer sequence, followed by a second enhancer sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second AAV ITR sequence.

In a non-limiting example of the preceding AAV piggyBac transposon polynucleotides, the at least one transgene sequence can comprise a nucleic acid sequence that encodes for a human phenylalanine hydroxylase (hPAH) polypeptide. In certain aspects, the nucleotide sequence encoding hPAH is codon optimized. In certain aspects, the codon optimized PAH gene is further modified to i) remove any internal TTAA sites; ii) any undesired restriction enzyme recognition sites; and iii) putative cryptic splice sites for cloning the modified sequences into AAV piggyBac transposon vectors. This non-limiting example of an AAV piggyBac transposon polynucleotide is shown in FIG. 2.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 18.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 21.

### AAV ITR Sequences

In some aspects, an AAV ITR sequence can comprise any AAV ITR sequence known in the art. In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 1 and 16.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 16.

piggyBac ITR Sequences

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 2 and 14.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 14.

In some aspects of the methods of the present disclosure, a piggyBac ITR sequence, such as a first piggyBac ITR sequence and/or a second piggyBac ITR sequence in an AAV piggyBac transposon can comprise, consist essentially of, or consist of a Sleeping Beauty transposon ITR, a Helraiser transposon ITR, a Tol2 transposon ITR, a TcBuster transposon ITR or any combination thereof.

In some aspects, a piggyBac ITR sequence of the present disclosure can be flanked on either or both ends by at least one of the following sequences: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-T AAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′, 5′-TTCT-3′ and 5′-TTTT-3′. In some aspects, a piggyBac ITR sequence can be flanked by 5′-TTAA-3′. Thus, any AAV piggyBac transposon polynucleotide of the present disclosure can further comprise any one of: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-T AAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-C AAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′, 5′-TTCT-3′ and 5′-TTTT-3′ flanking a piggyBac ITR sequence.

Mutant piggyBac ITR sequences with at least one nucleic acid substitution relative to a wildtype ITR of a piggyBac transposon can provide increased transposition efficacy and efficiency. An exemplary mutant piggyBac ITR sequence contains a 35TCC mutation. Further exemplary mutant piggyBac ITR sequences include those disclosed in International Patent Application No. PCT/US2022/77544, the contents of which is incorporated herein by reference in its entirety.

In some aspects, a mutant piggyBac ITR sequence comprising the 35 TCC mutation can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO:19.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2 and a second piggyBac ITR sequence can comprise a 35TCC mutation, and comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 19.

piggyBac 3′UTR Sequences

In some aspects, a piggyBac 3′UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 3.

piggyBac 5′UTR Sequences

In some aspects, a piggyBac 5′UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 13.

### Insulator Sequences

In some aspects, an insulator sequence can comprise any insulator sequence known in the art. In some aspects, an insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 4 and 12.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 4 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 12.

### Promoter Sequences

In some aspects, a promoter sequence can comprise any promoter sequence known in the art. In some aspects, a promoter sequence can comprise any liver-specific promoter sequence known in the art.

In some aspects, a promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 7.

In some aspects, a promoter sequence can comprise a TTRm promoter sequence. A TTRm promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 7.

### Transgene Sequences

In some aspects, a transgene sequence can comprise a nucleic acid sequence that encodes for a human phenylalanine hydroxylase (hPAH) polypeptide. In some aspects, a nucleic acid sequence that encodes for a hPAH polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 9.

In some aspects, a transgene sequence can comprise a tag, for example a hemagglutinin (HA) tag. Such tags are well known in the art and useful for protein purification.

In some aspects, a transgene sequence can be codon optimized according to methods known in the art.

In some aspects, the nucleic acid sequence encoding a polypeptide (e.g. hPAH) can be a codon optimized nucleic acid sequence that encodes for the polypeptide. A codon optimized nucleic acid sequence encoding a polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the polypeptide.

In some aspects, SEQ ID NO:9 is a unique codon optimized nucleic acid sequence that can be included in the polynucleotides, vectors and compositions of the present disclosure.

In some aspects, a codon optimized nucleic acid sequence encoding a polypeptide, such as that put forth in SEQ ID NO: 9, can comprise no donor splice sites. In some aspects, a codon optimized nucleic acid sequence encoding a polypeptide can comprise no more than about one, or about two, or about three, or about four, or about five, or about six, or about seven, or about eight, or about nine, or about ten donor splice sites. In some aspects, a codon optimized nucleic acid sequence encoding a polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding the polypeptide. Without wishing to be bound by theory, the removal of donor splice sites in the codon optimized nucleic acid sequence can unexpectedly and unpredictably increase expression of the polypeptide in vivo, as cryptic splicing is prevented. Moreover, cryptic splicing may vary between different subjects, meaning that the expression level of the polypeptide comprising donor splice sites may unpredictably vary between different subjects.

In some aspects, a codon optimized nucleic acid sequence encoding a polypeptide, such as that put forth in SEQ ID NO: 9, can have a GC content that differs from the GC content of the wildtype human nucleic acid sequence encoding the polypeptide. In some aspects, the GC content of a codon optimized nucleic acid sequence encoding a polypeptide is more evenly distributed across the entire nucleic acid sequence, as compared to the wildtype human nucleic acid sequence encoding the polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding a polypeptide, such as that put forth in SEQ ID NO: 9, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding the polypeptide.

In some aspects, an at least one transgene sequence can be operatively linked to at least one promoter sequence present in the same polynucleotide.

polyA Sequences

In some aspects, a polyA sequence can comprise any polyA sequence known in the art. Non-limiting examples of polyA sequences include, but are not limited to, SV40 polyA sequences. In some aspects, an insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 11.

### DNA Spacer Sequences

In some aspects, a DNA spacer sequence can comprise, consist essentially of or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 15 and 20.

DNA spacer sequences can be located at any position within an AAV piggyBac transposon polynucleotide or an AAV piggyBac transposase polynucleotide. In some aspects, the DNA spacer sequence is positioned between the second piggyBac ITR and the 3′ AAV ITR.

### Enhancer Sequences

In some aspects, an enhancer sequence can comprise any enhancer sequence known in the art. In some aspects, an enhancer sequence can comprise any liver-specific enhancer sequence known in the art.

In some aspects, an enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 5 and 6.

In some aspects, an enhancer sequence can comprise a 3×hSERPINA1 enhancer sequence. A 3×hSERPINA enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5.

In some aspects, an enhancer sequence can comprise a TTR enhancer sequence. A TTR enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 6.

### 3′ UTR Sequences

In some aspects, a 3′ UTR sequence can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 10.

In some aspects of the preceding transposons, a first 3′ UTR sequence can be an AES-mtRNR 3′ UTR sequence. An AES-mtRNR 3′ UTR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 10.

### Intron Sequences

In some aspects, an intron sequence can comprise, consist essentially of or consist of a 100% (or any percentage in between) identical to any one of the sequences put forth in SEQ ID NOs: 8 and 17.

In some aspects, an intron sequence can comprise an MVM intron sequence. An MVM intron sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 8.

In some aspects, an intron sequence can comprise a synthetic intron sequence. A synthetic intron sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 17.

### Transposase Sequences

In some aspects, a transposase sequence can comprise a nucleic acid sequence that encodes for any transposase polypeptide known in the art. In some aspects, a transposase sequence can comprise a nucleic acid sequence that encodes for a piggyBac® (PB) transposase polypeptide. In some aspects, a transposase sequence can comprise a nucleic acid sequence that encodes for a piggyBac-like (PBL) transposase polypeptide. In some aspects, a transposase sequence can comprise a nucleic acid sequence that encodes for a Super piggyBac® (SPB) transposase polypeptide.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′ TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-T AAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-AT AG-3′, 5′-C AAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 22.

The PB or PBL transposase can comprise or consist of an amino acid sequence having an amino acid substitution at two or more, at three or more or at each of positions 30, 165, 282, and/or 538 of the sequence of SEQ ID NO: 22. The transposase can be a SPB transposase that comprises or consists of the amino acid sequence of the sequence of SEQ ID NO: 22 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 23.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 22 or SEQ ID NO: 23 are described in more detail in PCT Publication No. WO 2019/173636 and PCT/US2019/049816.

In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 24.

The PB or PBL transposase can comprise or consist of an amino acid sequence having an amino acid substitution at two or more, at three or more or at each of positions 29, 164, 281, and/or 537 of the sequence of SEQ ID NO: 24. The transposase can be a SPB transposase that comprises or consists of the amino acid sequence of the sequence of SEQ ID NO: 24 wherein the amino acid substitution at position 29 can be a substitution of a valine (V) for an isoleucine (I), the amino acid substitution at position 164 can be a substitution of a serine(S) for a glycine (G), the amino acid substitution at position 281 can be a substitution of a valine (V) for a methionine (M), and the amino acid substitution at position 537 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 25.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 24 or SEQ ID NO: 25 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. B ADI 1135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, a PB, PBL or SPB transposase can fused to a nuclear localization signal. Examples of PB, PBL or SPB transposases fused to a nuclear localization signal are disclosed in U.S. Pat. Nos. 6,218,185; 6,962,810, 8,399,643 and WO 2019/173636. A nuclear localization signal can comprise, consist essentially of or consist of a of the amino acid sequence at least 65%, 70%, 75%, 80%, 85%, 90%, 95%, 96%, 97%, 98%, 99% or 100% (or any percentage in between) identical to SEQ ID NO: 26. A nuclear localization signal can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 27.

In some aspects, a nuclear localization signal can be fused to a PB, PBL or SPB transposase using a G4S linker located between the NLS and the PB, PBL or SPB. A G4S linker can comprise, consist essentially of or consist of an amino acid sequence at least 65%, 70%, 75%, 80%, 85%, 90%, 95%, 96%, 97%, 98%, 99% or 100% (or any percentage in between) identical to SEQ ID NO: 28. A G4S linker can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 29.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 30. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NO: 31.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 32. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NO: 33.

In some aspects, a transposase sequence can comprise a nucleic acid sequence that encodes for a Sleeping Beauty transposase polypeptide (for example as disclosed in U.S. Pat. No. 9,228,180). In some aspects, a transposase sequence can comprise a nucleic acid sequence that encodes for a Hyperactive Sleeping Beauty (SB100X) transposase polypeptide. In some aspects, a Sleeping Beauty transposase comprises or consists of an amino acid sequence at least 65%, 70%, 75%, 80%, 85%, 90%, 95%, 96%, 97%, 98%, 99% or 100% (or any percentage in between) identical to SEQ ID NOs: 34 or 35. In a preferred aspect, hyperactive Sleeping Beauty (SB100X) transposase comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NOs: 36 or 37.

In some aspects, a transposase sequence can comprise a nucleic acid sequence that encodes for a helitron transposase polypeptide (for example, as disclosed in WO 2019/173636). In some aspects, a Helitron transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NOs: 38 or 39.

In some aspects, a transposase sequence can comprise a nucleic acid sequence that encodes for a Tol2 transposase polypeptide (for example, as disclosed in WO 2019/173636). In some aspects, a Tol2 transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NOs: 40 or 41.

In some aspects, a transposase sequence can comprise a nucleic acid sequence that encodes for a TeBuster transposase polypeptide (for example, as disclosed in WO 2019/173636) or a mutant TcBuster transposase polypeptide (as described in more detail in PCT Publication No. WO 2019/173636 and PCT/US2019/049816). In some aspects, a TeBuster transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NOs: 42 or 43. The polynucleotide encoding a TcBuster transposase can comprise or consist of a naturally occurring nucleic acid sequence or a non-naturally occurring nucleic acid sequence.

### Vectors of the Present Disclosure

The present disclosure provides compositions comprising a vector, wherein the vector comprises at least one adeno-associated virus (AAV) piggyBac transposon polynucleotide. A vector comprising at least one adeno-associated virus (AAV) piggyBac transposon polynucleotide is herein referred to as an “AAV piggyBac transposon vector”.

The present disclosure provides compositions comprising a vector, wherein the vector comprises at least one AAV transposase polynucleotide. A vector comprising at least one AAV transposase polynucleotide is herein referred to as an “AAV transposase vector”.

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV).

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10 and AAV11). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, AAV8, AAV9, rAAV-LK03, AAV-KP-1 (also referred to as AAV-KP1; described in detail in Kerun et al. JCI Insight, 2019; 4 (22): el31610) and AAV-NP59 (described in detail in Paulk et al. Molecular Therapy, 2018; 26 (1): 289-303).

The present disclosure provides a composition comprising a plurality of AAV-KP-1 particles comprising at least one adeno-associated virus (AAV) piggyBac transposon polynucleotide. The present disclosure provides a composition comprising a plurality of AAV-NP59 particles comprising at least one adeno-associated virus (AAV) piggyBac transposon polynucleotide.

The present disclosure provides a composition comprising a plurality of AAV-NP59 (described in detail in Paulk et al. Molecular Therapy, 2018; 26 (1): 289-303).

The present disclosure provides a composition comprising a plurality of AAV8 particles comprising at least one adeno-associated virus (AAV) piggyBac transposon polynucleotide and a LNP composition comprising an mRNA encoding a transposase. In certain aspects, the mRNA molecule further comprises a 5′-CAP. In certain aspects, the transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase. In certain aspects, the transposase is a Super piggyBac™ (SPB) transposase. In certain aspects, the at least one LNP composition comprises: about 54% of ssPalmO-Ph-P4C2 by moles, about 35% of cholesterol by moles, about 10% of DOPC by moles, and about 1% of DMG-PEG2000 by moles.

The present disclosure provides a composition comprising a plurality of AAV9 particles comprising at least one adeno-associated virus (AAV) piggyBac transposon polynucleotide and a LNP composition comprising an mRNA encoding a transposase. In certain aspects, the mRNA molecule further comprises a 5′-CAP. In certain aspects, the transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase. In certain aspects, the transposase is a Super piggyBac™ (SPB) transposase. In certain aspects, the at least one LNP composition comprises: about 54% of ssPalmO-Ph-P4C2 by moles, about 35% of cholesterol by moles, about 10% of DOPC by moles, and about 1% of DMG-PEG2000 by moles.

The viral vectors and viral particles of the present disclosure can be produced using standard methods known in the art.

The cell delivery compositions (e.g., polynucleotides, vectors) disclosed herein can comprise a nucleic acid encoding a therapeutic protein or therapeutic agent. Examples of therapeutic proteins include those disclosed in PCT Publication No. WO 2019/173636 and PCT/US2019/049816. Therapeutic proteins can also include, but are not limited to, any one of polypeptides described herein as part of transgene sequences (e.g. hPAH).

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998.

Pharmaceutically acceptable carriers can be routinely selected that are suitable for the mode of administration, solubility and/or stability of the protein scaffold, fragment or variant composition as well known in the art or as described herein.

For example, the disclosed LNP compositions of the present disclosure can further comprise a diluent. In some compositions, the diluent can be phosphate buffered saline (“PBS”). In some compositions, the diluent can be sodium acetate.

Non-limiting examples of pharmaceutical excipients and additives suitable for use include proteins, peptides, amino acids, lipids, and carbohydrates (e.g sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (EISA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffmose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffmose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-P-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g, cholesterol), and chelating agents (e.g, EDTA).

Many known and developed modes can be used for administering therapeutically effective amounts of the compositions or pharmaceutical compositions disclosed herein. Non limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water. Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpoly ethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffmose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g, inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g, water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g, jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 pm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g, suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g, a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g, gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopocia, Tarascon Pocket Pharmacopoeia 2000, Deluxe Edition, Tarascon Publishing, Loma Linda, Calif. (2000); Nursing 2001 Handbook of Drugs. 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g, bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

Any use or method of the present disclosure can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

### Methods of the Present Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method of treating phenylketonuria (PKU) in a subject in need thereof comprising administering to the subject at least one therapeutically effective dose of a composition comprising: a) the polynucleotide, vector or pharmaceutical composition of any one of the preceding claims; and b) at least one LNP composition comprising at least one mRNA molecule encoding a transposase. In certain aspects, the mRNA molecule further comprises a 5′-CAP. In certain aspects, the transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase. In certain aspects, the transposase is a Super piggyBac™ (SPB) transposase. In certain aspects, the at least one LNP composition comprises: about 54% of ssPalmO-Ph-P4C2 by moles, about 35% of cholesterol by moles, about 10% of DOPC by moles, and about 1% of DMG-PEG2000 by moles.

The present disclosure provides at least one composition of the present disclosure for the use in the treatment of PKU in a subject, wherein the at least one composition is for administration to the subject in at least one therapeutically effective amount.

The present disclosure provides the use of at least one composition of the present disclosure for the manufacture of a medicament for the treatment of PKU in a subject, wherein the at least one composition is for administration to the subject in at least one therapeutically effective amount.

The present disclosure provides a method of treating at least one disease in a subject, the method comprising administering to the subject at least one therapeutically effective amount of at least one composition of the present disclosure comprising at least one nucleic acid encoding a therapeutic protein.

In some aspects, a nucleic acid molecule formulated in a composition of the present disclosure can comprise at least one transgene sequence. In some aspects, a transgene sequence can comprise a nucleotide sequence encoding at least one therapeutic protein.

In some aspects, a nucleic acid molecule formulated in a composition of the present disclosure comprising AAV piggyBac transposon polynucleotides can comprise at least one transgene sequence. In some aspects, a transgene sequence can comprise a nucleotide sequence encoding at least one therapeutic protein. In some aspects, a transgene sequence can comprise a nucleotide sequence encoding at least one transposon.

In some aspects, an at least one sequence encoding at least one therapeutic protein can be a sequence encoding a human phenylalanine hydroxylase (hPAH) polypeptide, wherein the hPAH polypeptide comprises the nucleic acid sequence of SEQ ID NO: 9. In certain aspects, the nucleotide sequence encoding hPAH is codon optimized.

In some aspects, a hPAH polypeptide comprises, consists essentially of or consist of a 100% (or any percentage in between) identical to SEQ ID NO: 9.

In some aspects of the preceding methods, a composition comprising a nucleic acid molecule comprising a nucleotide sequence encoding at least one transposase can be a composition comprising at least one LNP of the present disclosure, wherein the LNP comprises at least one nucleic acid molecule comprising a nucleotide sequence encoding at least one transposase. Accordingly, the present disclosure provides methods of treating at least one disease in a subject, the methods comprising administering to the subject: a) at least one therapeutically effective amount of a composition comprising a nucleic acid comprising a transposon, wherein the transposon comprises a nucleotide sequence encoding at least one therapeutic protein; and b) at least one therapeutically effective amount of LNPs of the present disclosure, wherein the LNPs comprise at least one nucleic acid comprising a nucleotide sequence encoding at least one transposase.

In some aspects of the preceding methods, a composition comprising a nucleic acid molecule comprising a nucleotide sequence encoding at least one transposon can be a composition comprising Adeno-associated virus (AAV) viral vector particles comprising at least one nucleic acid molecule comprising a transposon, wherein the transposon comprises a nucleotide sequence encoding at least one therapeutic protein. Accordingly, the present disclosure provides methods of treating at least one disease in a subject, the methods comprising administering to the subject: a) at least one therapeutically effective amount of AAV viral vector particles comprising at least one nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase. In certain methods, the nucleic acid molecule comprising a nucleotide sequence encoding at least one transposase is an mRNA.

In some aspects of the preceding methods, a composition comprising AAV viral vector particles can comprise at least one AAV piggyBac transposon polynucleotide. Accordingly, the present disclosure provides methods of treating at least one disease in a subject, the methods comprising administering to the subject: a) at least one therapeutically effective amount of a composition of AAV viral vector particles comprising at least one AAV piggyBac transposon polynucleotide and at least one nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

Additionally, the present disclosure provides methods of treating at least one disease in a subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one nucleic acid molecule comprising a nucleotide sequence encoding at least one transposase. In certain methods, the nucleic acid molecule comprising a nucleotide sequence encoding at least one transposase is an mRNA.

In some aspects of the preceding methods, a composition comprising AAV viral vector particles can comprise at least one AAV piggyBac transposon polynucleotide. Accordingly, the present disclosure provides methods of treating at least one disease in a subject, the methods comprising administering to the subject: a) at least one therapeutically effective amount of a composition of AAV viral vector particles comprising at least one AAV piggyBac transposon polynucleotide and at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one nucleic acid molecule comprising a nucleotide sequence encoding at least one transposase. In certain methods, the nucleic acid molecule comprising a nucleotide sequence encoding at least one transposase is an mRNA.

In a non-limiting example, AAV viral vector particles comprising at least one nucleic acid molecule comprising a transposon, wherein the transposon comprises a nucleotide sequence encoding at least one therapeutic protein, wherein the therapeutic protein is hPAH, can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of SEQ ID NOs: 18 and 21.

In some aspects of the preceding methods, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered concurrently. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered sequentially. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered in temporal proximity.

As used herein, the term “temporal proximity” refers to that administration of one therapeutic composition (e.g., a composition comprising a transposon) occurs within a time period before or after the administration of another therapeutic composition (e.g., a composition comprising a transposase), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In a non-limiting example, the present disclosure provides methods of treating PKU in a subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the at least one therapeutic protein can comprise human phenylalanine hydroxylase (hPAH) polypeptide.

In some aspects of the treatment methods of the present disclosure, the administration of the at least one composition and/or nanoparticle of the present disclosure to a subject can result in the expression of an exogenous protein (e.g. a therapeutic protein, a transposase, etc.) in at least one organ and/or tissue in the subject.

In some aspects, the administration of the at least one composition and/or nanoparticle of the present disclosure results in the expression of the exogenous protein in at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of the cells in the tissue and/or organ.

In some aspects, the administration of the at least one composition and/or nanoparticle of the present disclosure results in the expression of the exogenous protein in at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of a specific subset or subsets of cells in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in the tissue and/or organ.

In some aspects, the administration of the at least one composition and/or nanoparticle of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in a specific subset or subsets of cells in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in the tissue and/or organ.

In some aspects, the administration of the at least one composition and/or nanoparticle of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in a specific subset or subsets of cells in the tissue and/or organ.

The present disclosure provides methods of treating at least one disease in a subject, wherein the subject has a missense mutation (F263S) that inactivates the PAH gene (Pah^{en2}). Pah^{enu2} subjects exhibit classic PKU, with elevated blood phenylalanine (Phe) levels, cognitive deficiencies, and maternal PKU syndrome. See, for example, Charron C. et al., Molecular Therapy Vol. 11, Supplement 1, May 2005, S163-S164.

In some aspects, the present disclosure provides methods of treating PKU in a Pah^{enu2} subject, the methods comprising administering to the subject: a) at least one therapeutically effective amount AAV viral vector particles comprising at least one nucleic acid molecule comprising a transposon, wherein the transposon comprises a nucleotide sequence encoding at least one therapeutic protein; and b) at least one therapeutically effective amount of LNPs of the present disclosure, wherein the LNPs comprise at least one RNA molecule comprising a nucleotide sequence encoding at least one transposase. In some aspects, the at least one therapeutic protein can comprise human phenylalanine hydroxylase (hPAH) polypeptide.

### Cells and Modified Cells of the Disclosure

Cells and modified cells of the disclosure can be mammalian cells. Preferably, the cells and modified cells are human cells. In one aspect, the cells targeted for modification using the LNP compositions of the present disclosure are hepatocytes, a hepatic stellate cells, Kupffer cells or liver sinusoidal endothelial cells. In one embodiment, the LNP compositions comprise at least one mRNA molecule encoding a transposase and the modified cells are generated in vivo. In one embodiment, the LNP compositions comprise at least one DNA molecule encoding a transposon and the modified cells are generated in vivo. In one embodiment, the transposon comprises a nucleotide sequence encoding a therapeutic gene operatively linked to a liver-specific promoter.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engrafiment following administration to a subject. Allogeneic cells may be any type of cell. Allogenic cells can be stem cells or can be derived from stem cells. Allogeneic cells can be differentiated somatic cells.

### Nucleic Acid Molecules

Nucleic acid molecules of the disclosure encoding a therapeutic protein can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified enzymatically active portion of a therapeutic protein; nucleic acid molecules comprising the coding sequence for a therapeutic protein and nucleic acid molecules which comprise a nucleotide sequence substantially different from those described above but which, due to the degeneracy of the genetic code, still encode the therapeutic protein as described herein and/or as known in the art. Of course, the genetic code is well known in the art. Thus, it would be routine for one skilled in the art to generate such degenerate nucleic acid variants that code for a specific protein scaffold of the present disclosure. See, e.g., Ausubel, et al., supra, and such nucleic acid variants are included in the present disclosure.

As indicated herein, nucleic acid molecules of the disclosure which comprise a nucleic acid molecule encoding a therapeutic protein can include, but are not limited to, those encoding the amino acid sequence of an enzymatically active fragment of a therapeutic protein, by itself; the coding sequence for the entire a therapeutic protein or a portion thereof; the coding sequence for a therapeutic protein, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a therapeutic protein can be fused to a marker sequence, such as a sequence encoding a peptide that facilitates purification of the fused therapeutic protein.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra, or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra, or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra, or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure, host cells that are genetically engineered with the recombinant vectors, and the production of at least one therapeutic protein by recombinant techniques, as is well known in the art. See, e.g., Sambrook, et al., supra, Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD 19, CD271, CD34, CD22, CD20, CD33, CD52, or any combination thereof. Cell surface markers further include the suicide gene marker RQRS (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid molecule encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g, ATCC CRL 1650), COS-7 (e.g. ATCC CRL-1651), HEK293, BHK21 (e.g, ATCC CRL-10), CHO (e.g, ATCC CRL 1610) and BSC-1 (e.g, ATCC CRL-26) cell lines, Cos-7 cells, CHO cells, hep G2 cells. P3X63Ag8.653, SP2/0-Agl4, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphogly cerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. Sec, e.g., Ausubel et al., supra, Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts. B., et al., Molecular Biology of The Cell, Third Ed., Garland Publishing, Inc., New York, 1994). A therapeutic protein of the disclosure can include one or more amino acid substitutions, deletions or additions, from spontaneous or mutations and/or human manipulation, as specified herein. Amino acids in a therapeutic protein of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for maintaining the activity of the therapeutic protein can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active therapeutic protein of the disclosure. Biologically active therapeutic protein have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to therapeutic proteins and fragments, as described herein, which are modified by the covalent attachment of an organic moiety. Such modification can produce a protein scaffold fragment with improved pharmacokinetic properties (e.g increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120.000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified therapeutic proteins and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, poly lysine is more soluble in water than in octane. Thus, a therapeutic protein modified by the covalent attachment of polylysine is encompassed by the disclosure. Hydrophilic polymers suitable for modifying therapeutic proteins of the disclosure can be linear or branched and include, for example, polyalkane glycols (e.g., PEG, monomethoxy-poly ethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the therapeutic protein of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying therapeutic proteins of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-A9-octadecanoate (C18, oleate), all cis-Δ5.8.1 1.14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified therapeutic proteins and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified therapeutic proteins of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified therapeutic proteins and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnok Bioeng., 56 (4): 456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system.

For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

“Binding” refers to a sequence-specific, non-covalent interaction between macromolecules (e.g between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten-to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter. CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 1.

TABLE 1
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY. N.Y. (1975). pp. 71-77) as set forth in Table 2.

TABLE 2
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 3.

TABLE 3
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring.

Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1—Construction of Exemplary AAV piggyBac Transposon Polynucleotides comprising a Codon Optimized and Modified Human Phenylalanine Hydroxylase (PAH) Gene

The following is a non-limiting example demonstrating the construction of exemplary AAV piggyBac transposon polynucleotides comprising a codon optimized and modified human PAH Gene.

The nucleotide sequence of the mRNA encoding human PAH sequence (GenBank U49897.1) was codon optimized to improve PAH expression using computer algorithms in accordance with the various manufacturer's instructions (e.g., ThermoFisher, Blue Heron Biotech, and Integrated DNA Technologies). After codon optimization, the optimized sequence was further modified to: i) remove any internal TTAA sites; ii) any undesired restriction enzyme recognition sites; and iii) putative cryptic splice sites for cloning the modified sequences into AAV piggyBac transposon vectors.

Two AAV piggyBac transposon polynucleotides comprising the codon optimized and modified human PAH gene were constructed. A general schematic diagram of each construct is shown in FIGS. 1 and 2, respectively.

As shown in FIG. 1, the first AAV piggyBac transposon polynucleotide comprises in the 5′ to 3′ direction: a) a first AAV ITR sequence (SEQ ID NO. 1); b) a first piggyBac ITR sequence (SEQ ID NO. 2); c) a piggyBac 3′ UTR sequence (SEQ ID NO. 3); d) a first insulator sequence (SEQ ID NO. 4); e) a TTRm promoter sequence (SEQ ID NO. 7); f) a synthetic intron sequence (SEQ ID NO. 17); g) a transgene sequence comprising a nucleic acid sequence encoding a hemagglutinin (HA)-tagged, codon optimized and modified human PAH gene (SEQ ID NO. 9); h) a polyA sequence (SEQ ID NO. 11); i) a second insulator sequence (SEQ ID NO. 12); j) a piggyBac 5′ UTR sequence (SEQ ID NO. 13) k) a second piggyBac ITR (SEQ ID NO. 14); l) a DNA spacer sequence (SEQ ID NO. 15); and m) a second AAV ITR sequence (SEQ ID NO. 16).

The first AAV piggyBac transposon polynucleotide was constructed using Gibson assembly from a single DNA fragment comprising the TTRm promoter-HA-PAH gene-polyA; and ii) a vector backbone containing insulator sequences, piggyBac ITRs and UTRs flanked by AAV ITRs. The complete nucleotide sequence of the first AAV piggyBac transposon polynucleotide is provided in SEQ ID NO. 18.

As shown in FIG. 2, the second AAV piggyBac transposon polynucleotide comprises in the 5′ to 3′ direction: a) a first AAV ITR sequence (SEQ ID NO. 1); b) a first piggyBac ITR sequence (SEQ ID NO. 2); c) a piggyBac 3′ UTR sequence (SEQ ID NO. 3); d) a first insulator sequence (SEQ ID NO. 4); e) a TTRm promoter region comprising a 3×hSERPINA1 enhancer (SEQ ID NO. 5), a TTR enhancer (SEQ ID NO. 6), a TTRm promoter sequence (SEQ ID NO. 7), and a MVM intron (SEQ ID NO. 8); f) a transgene sequence comprising a nucleic acid sequence encoding a hemagglutinin (HA)-tagged, codon optimized and modified human PAH gene (SEQ ID NO. 9) followed by a AES-mtRNR 3′ UTR (SEQ ID NO. 10): g) a polyA sequence (SEQ ID NO. 11); h) a second insulator sequence (SEQ ID NO. 12; i) a piggyBac 5′ UTR sequence (SEQ ID NO. 13) a second piggyBac ITR sequence comprising a 35TCC mutation (SEQ ID NO. 19); j) a DNA spacer sequence (SEQ ID NO. 20); and k) a second AAV ITR sequence (SEQ ID NO. 16).

The second AAV piggyBac transposon polynucleotide was constructed using Gibson assembly by combining a DNA fragment comprising the TTRm promoter-HA-PAH gene-polyA sequences; a DNA fragment containing the piggyBac 5′ ITR with the 35TCC variant; and ii) a vector backbone containing insulator sequences, piggyBac ITRs and 3′ UTR flanked by AAV ITRs. The complete nucleotide sequence of the second AAV piggyBac transposon polynucleotide is provided in SEQ ID NO. 21.

### Example 2—Preparation of 5′-Capped mRNA Encoding Super PiggyBac Transposase (“SPB”) for Encapsulation in LNP Compositions

The following is a non-limiting example demonstrating the preparation of an exemplary mRNA encoding SPB that may be incorporated in LNP compositions for use in combination in methods with AAV piggyBac transposon polynucleotides and vectors of the present disclosure for treating PKU.

The DNA plasmid pRTb_HBB_SPBv3.1 encodes SPB transposase comprising an N-terminal SV40 nuclear localization signal (NLS) and containing the 5′ and 3′ UTRs of the human beta-globin gene (HBB).

This plasmid was used as a template for in vitro transcription reactions to produce mRNA encoding HBB-SPBv3.1 further comprising a 5′-CAP.

| Briefly, approximately 300 μg of supercoiled pRTb_HBB_SPBv3.1 was added to a 15 mL conical comprising 300 μL CutSmart Buffer, 60 μL of the restriction enzyme Bbsl-HF (New England Biolabs, Cat #R3539M) in 3000 μL total volume. The plasmid DNA was linearized by incubating at 37° C. overnight to ensure complete digestion.

The linearized plasmid was purified using a DNA QIAquick PCR purification kit (Qiagen, Cat #28106) according to the manufacturer's instructions, and the purified DNA eluted in 900 μL of nuclease-free water (ThermoFisher, Cat #AM9937). The DNA concentration and purity of the eluate was determined using a NanoDrop microvolume spectrophotometer (ThermoFisher) in accordance with the manufacturer's instructions.

The purified plasmid was used as a DNA template to produce mRNA using the custom in vitro transcription mMESSAGE mMACHINE T7 Transcription Kit (ThermoFisher, Cat #AM1345B001) in accordance with internal, Quality-controlled manufacturing batch records. Briefly, 100 mM stocks of the nucleotides GTP (ThermoFisher, Cat #R0481), ATP (ThermoFisher, Cat #R0481), UTP (ThermoFisher, Cat #R0481), and 5MeC (5-Methylcytidine-5′-Triphosphate) (TriLink, Cat #N-1014) and CleanCap Reagent AG (m7G(5′)ppp(5′)(2′OMeA)pG; Trilink, Cat #N-7113) were prepared. 1,485 μL each of ATP, UTP and 5MeC and 1,188 μL each of GTP and CleanCap Reagent AG were blended.

153 μg of linearized pRTb_HBB_SPBv3.1 DNA, 1,800 μL of 10× T7 RXN Buffer (ThermoFisher, Cat #AM1345B001), 1,800 μL of T7 Enzyme mix (ThermoFisher, Cat #AM1345B001), and 6,831 μL of the NTP and cap blend were added to a 50 ml conical (18,000 AL final volume), and incubated at 37° C. for 3 hours. A 900 AL aliquot of DNaseI enzyme (ThermoFisher, Cat #AM1345B001) was added and the tube further incubated at 37° C. for 15 min to degrade the DNA template.

A poly(A) tail was post-enzymatically added to the 3′ end of the 5′-CleanCap®-HBB-SPBv3.1-5MeC mRNA. 18,000 μL of 5×EPAP Buffer (ThermoFisher, Cat #AM1345B001), 9,000 μL of 25 mM MnCl₂ (ThermoFisher, Cat #AM1345B001), 9,000 μL ATP Solution (ThermoFisher, Cat #AM1345B001), and 3,000 μL E-PAP (ThermoFisher, Cat #AM1345B001), were added to the IVT reaction (90,000 μL total volume), and incubated at 37° C. for 1 hour. The bulk E-PAP reaction was subsequently divided into three 125 mL PETG bottles in 30 mL aliquots.

The 5′-CleanCap®-HBB-SPBv3.1-poly(A)-5MeC mRNA was purified using a RNeasy Maxi Purification Kit (Qiagen, Cat #75162) according to the manufacturer's instructions. Briefly, a working stock of Buffer RLT was formulated using 178.2 mL of Buffer RLT (Qiagen, Cat #75162) with 1,800 μL of 2-mercaptoethanol (Sigma, Cat #M3148). 52.2 mL of the BME+RLT solution and 37.8 mL of 100% EtOH (ThermoFisher, Cat #BP2818) were added to each 30 mL mRNA aliquot. The purified mRNA product was eluted in 52.5 mL of nuclease-free water, and the bulk product stored at −80° C. The DNA linearization, IVT, and mRNA purification process is repeated until the target yield is reached.

Bulk mRNA lots were analyzed using gel electrophoresis before combination in a 500 mL PETG bottle, and sampled for concentration readings using the NanoDrop. Lithium Chloride 5× (ThermoFisher, Cat #AM1345B001) was added to the pooled mRNA in the amount of ⅓ of the total volume of the mRNA, then divided into equal 40 mL aliquots in 50 mL conical tubes and incubated at −20° C. for 45 minutes. Directly following incubation, the conical tubes are centrifuged at 14,000 g for 30 minutes at 4° C. The mRNA pellet is washed using 70% EtOH (ThermoFisher, Cat #BP8201) three times.

The washed mRNA pellets are dried, then resuspended in nuclease-free water. The mRNA concentration was determined using the NanoDrop, and additional nuclease-free water was added as necessary to further dilute the product to the target concentration. The mRNA is sterile filtered using a 0.22 μm PES SteriCup Filter (Sigma, Cat #52GPU05RE) before the final mRNA concentration and purity are measured on the NanoDrop.

### Example 3—Preparation of an LNP Composition Comprising a 5′-Capped mRNA Encoding Super PiggyBac Transposase

The following is a non-limiting example that provides exemplary methods for formulating an LNP composition comprising a 5′-capped mRNA encoding SPB transposase for use in combination with the AAV piggyBac transposon vectors disclosed herein.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages.

A 1 mg/ml solution of the 5′-CleanCap-5MeC-SPB mRNA prepared in Example 2 to be incorporated into the LNPs was added to 150 mM sodium acetate buffer (pH 5.2) to form a stock solution and kept on ice. The lipid phase was mixed with the aqueous mRNA phase inside a microfluidic chip using aNanoAssemblr® instrument (Precision Nanosystems, Vancouver, BC, Canada) according to the manufacturer's instructions to form LNP compositions comprising encapsulated SPB mRNAs. Nanoassemblr process parameters for mRNA encapsulation were at a flow rate of 20 ml/min and at a lipid:RNA ratio (v/v) of 1:3.

The resultant SPB mRNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against 25 mM sodium acetate (dialysate:dialysis buffer volume at least 1:200 v/v), pH 5.5 overnight at 4° C. (or alternatively room temperature for at least 4 hours), to remove the 25% ethanol and achieve a complete buffer exchange. In applicable, the LNP compositions were further concentrated using an Amicon® Ultra-4 centrifugal filter unit, MWCO-30 kDa (Millipore Sigma, USA) spun at ˜4100×g in an ultracentrifuge. Sucrose was added to a final concentration of 5% (w/v) to the mRNA LNPs which were then stored at 4° C. or frozen at −80° C. until further use. The average particle size diameter of the LNPs ranged from approximately 84-121 nm.

### Example 4—Compositions of the Present Disclosure for the Treatment of PKU

The following is a non-limiting example demonstrating the AAV piggyBac transposon polynucleotides and LNP-comprising compositions and methods of the present disclosure can be used in the treatment of PKU.

Adult (10-12 weeks old) C57BL/6 mice (n=3 mice per group) were intravenously administered the following treatments:
Treatment #1: Codon optimized and modified human phenylalanine hydroxylase (hPAH) transposon AAV viral vector particles;
Treatment #2: Human phenylalanine hydroxylase (hPAH) transposon AAV viral vector particles in combination with LNPs encapsulating mRNA encoding functional SPB transposase;
Treatment #3: Human phenylalanine hydroxylase (hPAH) transposon AAV viral vector particles in combination with LNPs encapsulating a catalytically inactive SPB transposase.

The human phenylalanine hydroxylase (hPAH) transposon AAV viral vector particles were AAV viral vector particles comprising an AAV piggyBac transposon polynucleotide comprising the nucleic acid of SEQ ID NO: 18.

The LNP composition comprising the mRNA encoding SPB transposase prepared in Example 2 comprised the following components: ssPalmO-Ph-P4C2, DOPC, Cholesterol and DMG-PEG2000 at a molar ratio of 54:10:35:1 and had a lipid:RNA ratio 100:1 (w/w).

The catalytically inactive mRNA SPB LNP compositions also comprised ssPalmO-Ph-P4C2, DOPC. Cholesterol and DMG-PEG2000 at a molar ratio of 54:10:35:1 and had a lipid:RNA ratio 100:1 (w/w).

For the Treatment #1 group, mice were administered either 1e12 GC/kg or 3e12 GC/kg of hPAH AAV piggyBac transposon vector, whereas mice in Treatment #2 group also were administered 0.5 mg/kg LNP compositions comprising an mRNA encoding SPB, and mice in Treatment #3 group also were administered 0.5 mg/kg LNP compositions comprising an mRNA encoding a catalytically inactive SPB.

After fourteen days post-administration, liver biopsies and blood samples were collected from treated mice. To isolate genomic DNA, liver samples were mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissue was then incubated at 56° C. for 30 minutes, and column-purified using a Monarch Genomic DNA Purification kit (New England Biolabs) in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a Nanodrop device. DNA samples were used for vector copy number (VCN) quantification.

To isolate proteins, liver samples were mixed with RIPA buffer and HALT protease inhibitor (both from ThermoFisher) and pulverized in a TissueLyser II (Qiagen) as described above. Lysates were then subjected to centrifugation for 10 mins at 20000 g and supernatants were collected for ELISA quantification of HA-tagged hPAH.

Liver biopsies were fixed in formalin for 24 hours, transferred to 70% ethanol for 24 hours, embedded in paraffin and processed for immunofluorescence detection of HA-tagged hPAH as described in Example 5.

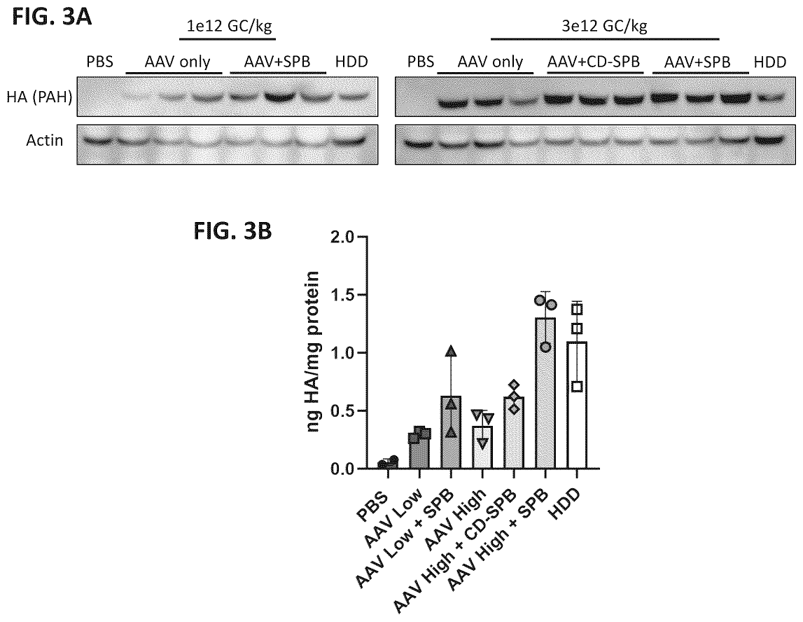

The results of the ELISA assay are described in FIGS. 3A and 3B. The expression of hPAH was shown to be dose dependent (FIG. 3A); and the addition of LNPs comprising SPB mRNA significantly increased hPAH protein expression compared to the levels observed in mice treated with AAV alone or hPAH-AAV co-delivered with a catalytically inactive SPB mutant. Expression levels of hPAH protein in mice co-administered the PAH AAV piggyBac transposon polynucleotide were similar to or exceeded those observed using hydrodynamic injection (FIG. 3B).

### Example 5—Immunofluorescence Staining

The number of transgene-positive hepatocytes in mice from the three treatment groups described in Example 4 were measured by immunofluorescence (IF) staining. Briefly, liver sections were incubated with an anti-HA primary antibody followed by a secondary antibody conjugated to a fluorescent dye. After nuclear counterstaining with DAPI, high-resolution whole slide digital images were obtained, and HA-positive cells were measured using an AI-powered image analysis algorithm (Reveal Biosciences).

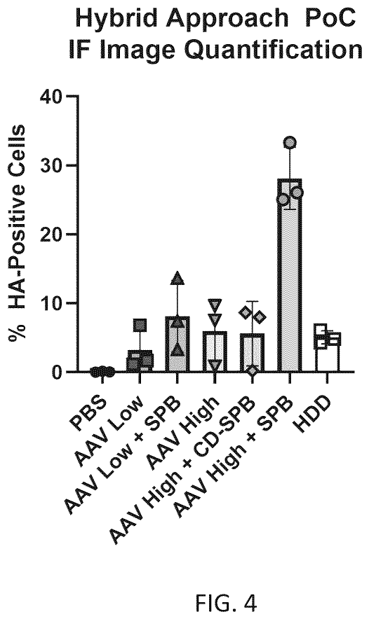

The results of the IF image quantification are shown in FIG. 4. The addition of active SPB increased the number of hPAH-expressing hepatocytes compared to the number of hPAH-expressing hepatocytes observed from mice treated with AAV alone or hPAH-AAV co-delivered with catalytically inactive SPB.

### Example 6—Determination of Episomal and Integrated Viral Copy Number

The levels of integrated viral copy number (VCN) in the liver of mice from the three treatment groups described in Example 4 were measured by a combination of three different digital droplet PCR (ddPCR) assays. Briefly, total copy number per cell was measured using an assay that binds to the transgene sequence, episomal copy number was measured using an assay that binds to the junction between backbone and transgene, and number of diploid genomes were measured using an assay that binds to the mouse HMBS genomic sequence. Then, VCN was calculated by subtracting episomal copy number from total copy number and expressed as integrated copies by diploid genome.

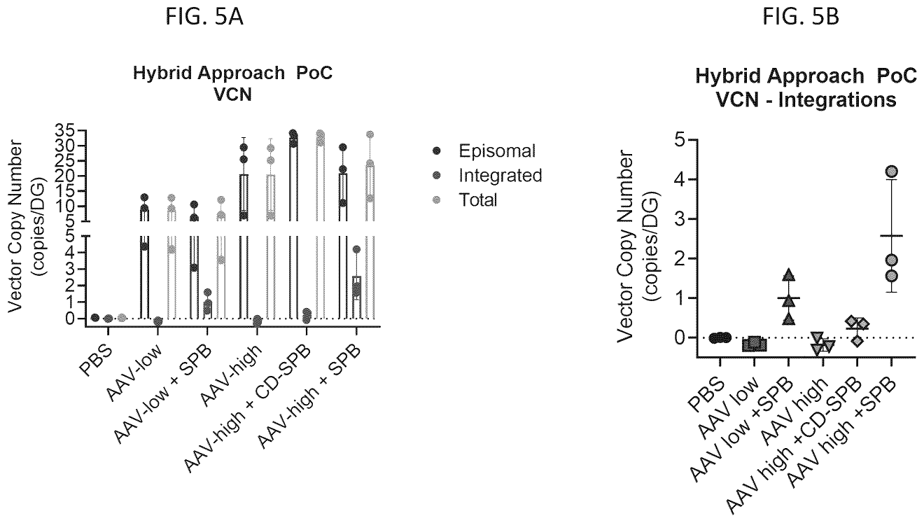

The results of this analysis are shown in FIGS. 5A and 5B. The numbers of total and integrated vectors increased with increasing AAV dose (FIG. 5A). Integration of viral vectors was only detected in mice treated with AAV co-delivered with catalytically active SPB.

Taken together, the results shown in Examples 4, 5 and 6 suggest that the transposon comprising the codon optimized and modified PAH gene was successfully integrated by catalytically active SPB into the host genome, leading to subsequent stable expression of PAH in transposed hepatocytes. In contrast, administration of the transposon comprising the transgene alone, or co-administration of the transposon with a catalytically inactive SPB transposase, showed little to no integration of the transgene and subsequently, less stable expression of PAH in hepatocytes.

### Example 7—Co-Administration of an AAV piggyBac Transposon Vector Comprising a Codon Optimized and Modified PAH Gene and an LNP Composition Comprising an mRNA Encoding a SPB Transposase Restores Normal Serum Phenylalanine Levels and Hypopigmentation in BTBR PAH^{enu} Mouse Model

The following is a non-limiting example demonstrating that co-administration of an AAV piggyBac transposon vectors comprising an AAV piggyBac transposon polynucleotide comprising a codon optimized and modified hPAH gene (Construct #1; SEQ ID NO. 18) and an LNP composition comprising an mRNA encoding a SPB transposase restores normal serum phenylalanine levels and normal hypopigmentation in a BTBR PAH^{enu} mouse model.

Adult male (n=1-3/group) and adult female (n=1-3/group) BTBR PAH^{enu} mice were divided into three groups. The first group was administered vehicle (PBS); the second group was co-administered 1e12 GC/kg of an AAV piggyBac transposon vector and 0.5 mg/kg of the ND4-2 LNP formulation of Example 3 comprising the 5′capped mRNA encoding SPB described in Example 2; and the third group was co-administered the ND4-2 LNP formulation of Example 3 comprising an mRNA encoding a catalytically-inactive version of SPB. Untreated adult C57BL/6 wild type mice (n=3/group) were followed in parallel as a positive control for measuring normal serum Phe levels.

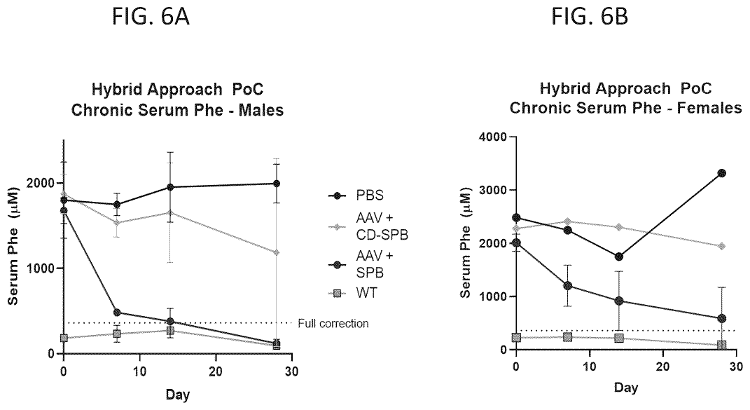

On Days 0, 7, 14 and 28, blood samples were drawn from each group of treated mice and control mice, and serum Phe levels were quantified using a colorimetric readout (Abcam). The results are shown in FIG. 6. As shown in FIG. 6A, male mice treated with an AAV piggyBac transposon PAH vector and the ND4-2 LNP formulation comprising the mRNA encoding SPB exhibited a rapid and steep decrease in serum Phe levels at Day 7 approaching wild type levels that continued to further decline below baseline at Days 14 & 28 (FIGS. 6A and 6B). Female mice also exhibited a reduction of serum Phe levels at Days 7 and 14, though to a lesser degree than males (FIG. 6B); however, at Day 28 serum Phe levels were reduced to levels observed for normal wild type mice.

In contrast, male and female mice treated with an AAV piggyBac transposon PAH vector and an LNP composition comprising a catalytically inactive SPB showed little to no decrease in serum Phe levels at Days 0, 7 and 14 and only a marginal reduction in serum Phe levels at Day 28 compared to mice treated with vehicle. Male and female mice treated with vehicle exhibited high levels of serum Phe levels that maintained over the course of the 28 days.

In addition, the degree of the restoration of hypopigmentation of treated and control mice was visually monitored for male and female mice. The observance of fur color hypopigmentation restoration in the BTBR PAH^{enu} mouse model is evidence of catabolism of serum Phe and subsequent restoration of coat melanin production. Male and female mice treated with an AAV piggyBac transposon PAH vector and the ND4-2 LNP formulation comprising the mRNA encoding SPB each exhibited a visual change in hypopigmentation coat color whereas male and female mice treated with an AAV piggyBac transposon PAH vector and an LNP composition comprising a catalytically inactive SPB or vehicle showed little or no visual change in coat color.

Thus, the co-administration of an AAV piggyBac transposon vector comprising a codon optimized PAH gene of the present disclosure and an LNP composition comprising an mRNA encoding SPB in an in vivo PKU disease model reduced serum Phe levels to levels equivalent or below that of wild type mice. In contrast, the same AAV piggyBac transposon vector co-administered with an LNP composition comprising an mRNA encoding a catalytically inactive SPB transposase showed little to no reduction of serum Phe levels that were similar to vehicle treated animals. These results suggest that the integration of the transposon comprising the codon optimized and modified PAH gene by SPB, and subsequent stable expression of PAH in transposed hepatocytes resulted in the reduction of serum Phe levels to normal wild type levels whereas co-administration of a catalytically inactive SPB transposase showed little to no reduction of serum Phe levels.

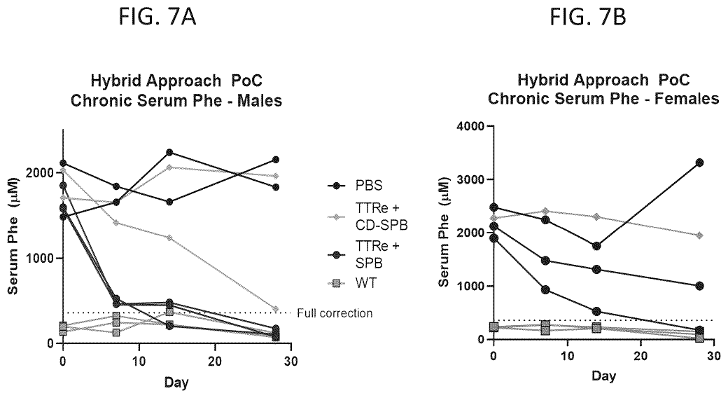

In another non-limiting example, co-administration of an AAV piggyBac transposon vectors comprising an AAV piggyBac transposon polynucleotide comprising a codon optimized and modified hPAH gene (Construct #2; SEQ ID NO. 21) and an LNP composition comprising an mRNA encoding a SPB transposase also restores normal serum phenylalanine levels and normal hypopigmentation in a BTBR PAH^{enu} mouse model (FIGS. 7A and 7B).

On Days 0, 7, 14 and 28, blood samples were drawn from each group of treated mice and control mice, and serum Phe levels were quantified using a colorimetric readout (Abcam). The results are shown in FIG. 7. As shown in FIG. 7A, male mice treated with an AAV piggyBac transposon PAH vector comprising Construct #2 and the ND4-2 LNP formulation comprising the mRNA encoding SPB exhibited a rapid and steep decrease in serum Phe levels at Day 7 approaching wild type levels that continued to further decline below baseline at Days 14 & 28 (FIG. 7A). Female mice also exhibited a reduction of serum Phe levels at Days 7 and 14, though to a lesser degree than males (FIG. 7B); however, at Day 28 serum Phe levels were reduced to levels observed for normal wild type mice.

In contrast, male and female mice treated with an AAV piggyBac transposon PAH vector and an LNP composition comprising a catalytically inactive SPB showed little to no decrease in serum Phe levels at Days 0, 7, or 14 in male or female mice and an appreciable reduction at Day 28 in male mice and only a marginal reduction in serum Phe levels at Day 28 in female mice compared to mice treated with vehicle. Male and female mice treated with vehicle exhibited high levels of serum Phe levels that maintained over the course of the 28 day period.

### Example 8—Compositions of the Present Disclosure for the Treatment of PKU

The following is a non-limiting example demonstrating the AAV piggyBac transposon polynucleotides and LNP-comprising compositions and methods of the present disclosure can be used in the treatment of PKU in a juvenile setting.

Juvenile (14 days old) C57BL/6 mice (n=3 mice per group) were intravenously administered the following treatments:
Treatment #1: Codon optimized and modified human phenylalanine hydroxylase (hPAH) transposon AAV viral vector particles in combination with LNPs encapsulating mRNA encoding functional SPB transposase;
Treatment #2: Codon optimized and modified human phenylalanine hydroxylase (hPAH) transposon AAV viral vector particles in combination with LNPs encapsulating a catalytically inactive SPB transposase.

A third group of mice was treated with vehicle alone.

The codon optimized and modified human phenylalanine hydroxylase (hPAH) transposon AAV viral vector particles were AAV viral vector particles comprising an AAV piggyBac transposon polynucleotide comprising the nucleic acid of SEQ ID NO: 18.

The LNP composition comprising the mRNA encoding SPB transposase prepared in Example 2 comprised the following components: ssPalmO-Ph-P4C2, DOPC, Cholesterol and DMG-PEG2000 at a molar ratio of 54:10:35:1 and had a lipid:RNA ratio 100:1 (w/w).

The catalytically inactive mRNA SPB LNP compositions also comprised ssPalmO-Ph-P4C2, DOPC, Cholesterol and DMG-PEG2000 at a molar ratio of 54:10:35:1 and had a lipid:RNA ratio 100:1 (w/w).

For the Treatment #1 group, mice were co-administered 1e12 GC/kg of hPAH AAV piggyBac transposon vector and 0.5 mg/kg LNP compositions comprising mRNA encoding functional SPB. Mice in the Treatment #2 group were co-administered 1e12 GC/kg of hPAH AAV piggyBac transposon vector and 0.5 mg/kg LNP compositions comprising mRNA encoding catalytically inactive SPB.

After four weeks post-administration, liver biopsies were collected from treated mice. To isolate genomic DNA, liver samples were mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissue was then incubated at 56° C. for 30 minutes, and column-purified using a Monarch Genomic DNA Purification kit (New England Biolabs) in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a Nanodrop device. DNA samples were used for vector copy number (VCN) quantification.

To isolate proteins, liver samples were mixed with RIPA buffer and HALT protease inhibitor (both from ThermoFisher) and pulverized in a TissueLyser II (Qiagen) as described above. Lysates were then subjected to centrifugation for 10 mins at 20000 g and supernatants were collected for ELISA quantification of HA-tagged hPAH.

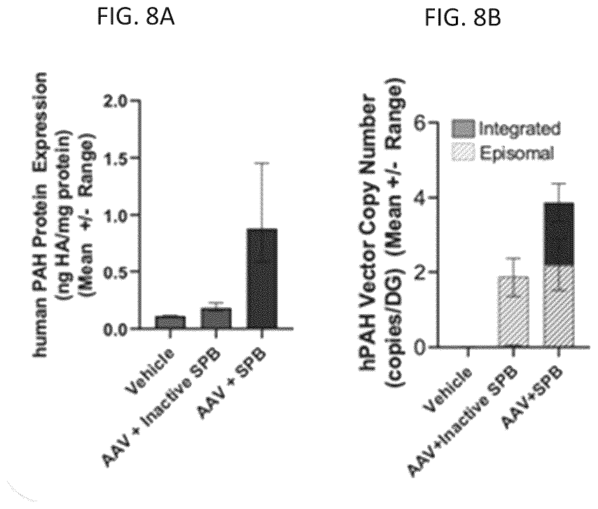

The results of the ELISA assay are described in FIGS. 8A and 8B. The addition of LNPs comprising functional SPB mRNA significantly increased hPAH protein expression compared to the levels observed in mice treated with hPAH-AAV co-delivered with a catalytically inactive SPB mutant (FIG. 8A).

The levels of integrated viral copy number (VCN) in the livers of mice from the two treatment groups were measured by digital droplet PCR (ddPCR) assays as described in Example 6. The results of this analysis are shown in FIG. 8B. Integration of viral vectors was only detected in mice treated with AAV co-delivered with catalytically active SPB.

The results in this Example demonstrate that SPB-mediated integration maintains PAH protein expression in a juvenile setting.

1. An adeno-associated virus (AAV) piggyBac transposon polynucleotide comprising in the 5′ to 3′ direction:
a) a first AAV ITR sequence;
b) a first piggyBac ITR sequence;
c) a first piggyBac 3′ UTR;
d) a first insulator sequence;
e) at least one promoter sequence;
f) at least one intron sequence;
g) at least one transgene sequence encoding human phenylalanine hydroxylase (PAH);
h) a polyA sequence;
i) a second insulator sequence;
j) a first piggyBac 5′ UTR sequence;
k) a second piggyBac ITR sequence;
l) at least one DNA spacer sequence; and
m) a second AAV ITR sequence.

2. An adeno-associated virus (AAV) piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 2;
c) a first piggyBac 3′ UTR sequence comprising the nucleic acid sequence of SEQ ID NO: 3;
d) a first insulator sequence comprising the nucleic acid sequence of SEQ ID NO: 4;
e) a promoter sequence comprising the nucleic acid sequence of SEQ ID NO: 7;
f) a synthetic intron sequence comprising the nucleic acid sequence of SEQ ID NO: 17;
g) a transgene sequence encoding a codon optimized and modified human PAH gene comprising the nucleic acid sequence of SEQ ID NO: 9;
h) a polyA sequence comprising the nucleic acid sequence of SEQ ID NO: 11;
i) a second insulator sequence comprising the nucleic acid sequence of SEQ ID NO: 12;
j) a piggyBac 5′UTR sequence comprising the nucleic acid sequence of SEQ ID NO: 13;
k) a second piggyBac ITR sequence comprising the nucleic acid sequence of SEQ ID NO: 14;
l) a DNA spacer sequence comprising the nucleic acid sequence of SEQ ID NO: 15; and
m) a second AAV ITR sequence comprising the nucleic acid sequence of SEQ ID NO: 16.

3. The AAV piggyBac transposon polynucleotide of claim 1 or 2, wherein the AAV piggyBac transposon polynucleotide comprises the nucleic acid sequence of SEQ ID NO: 18.

4. A vector comprising the AAV piggyBac transposon polynucleotide of any of the preceding claims.

5. The vector of claim 4, wherein the vector is an AAV viral vector.

6. The vector of claim 5, wherein the AAV viral vector is an AAV1, AAV2, AAV3, AAV4, AAV5, AAV6, AAV7, AAV8, AAV9, AAV10 or AAV11 viral vector.

7. The vector of claim 6, wherein the AAV viral vector is an AAV8 or AAV9 viral vector.

8. An adeno-associated virus (AAV) piggyBac transposon polynucleotide comprising in the 5′ to 3′ direction:
a) a first AAV ITR sequence;
b) a first piggyBac ITR sequence;
c) a first piggyBac 3′ UTR;
d) a first insulator sequence;
e) at least one enhancer sequence;
f) at least one promoter sequence;
g) at least one transgene sequence encoding human phenylalanine hydroxylase (PAH);
h) a first 3′ UTR;
i) a polyA sequence;
j) a second insulator sequence;
k) a first piggyBac 5 UTR;
l) a second piggyBac ITR sequence comprising a 35TCC mutation;
m) at least one DNA spacer sequence; and
n) a second AAV ITR sequence.

9. An adeno-associated virus (AAV) piggyBac transposon polynucleotide comprising in the 5′ to 3′ direction:
a) a first AAV ITR sequence;
b) a first piggyBac ITR sequence;
c) a first piggyBac 3′ UTR;
d) a first insulator sequence;
e) a 3× hSERPINA1 enhancer;
f) a TTR enhancer;
g) a TTRm promoter sequence;
h) a MVM intron;
i) a transgene sequence comprising a nucleic acid sequence encoding a codon optimized and modified human PAH gene;
j) a AES-mtRNR 3′ UTR;
k) a polyA sequence;
l) a second insulator sequence;
m) a first piggyBac 5′ UTR;
n) a second piggyBac ITR sequence comprising a 35TCC mutation;
o) a DNA spacer sequence; and
p) a second AAV ITR sequence.

10. An adeno-associated virus (AAV) piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 2;
c) a first piggyBac 3′ UTR sequence comprising the nucleic acid sequence of SEQ ID NO: 3;
d) a first insulator sequence comprising the nucleic acid sequence of SEQ ID NO: 4;
e) a first enhancer sequence comprising the nucleic acid sequence of SEQ ID NO: 5;
f) a second enhancer sequence comprising the nucleic acid sequence of SEQ ID NO: 6;
g) a promoter sequence comprising the nucleic acid sequence of SEQ ID NO: 7;
h) an intron sequence comprising the nucleic acid sequence of SEQ ID NO: 8;
i) a transgene sequence encoding a codon optimized and modified human PAH gene comprising the nucleic acid sequence of SEQ ID NO: 9;
j) an AES-mtRNR 3′ UTR comprising the nucleic acid of SEQ ID NO. 10;
k) a polyA sequence comprising the nucleic acid sequence of SEQ ID NO: 11;
l) a second insulator sequence comprising the nucleic acid sequence of SEQ ID NO: 12;
m) a piggyBac 5′UTR sequence comprising the nucleic acid sequence of SEQ ID NO: 13;
n) a second piggyBac ITR sequence comprising a 35TCC mutation, comprising the nucleic acid sequence of SEQ ID NO: 19;
o) a DNA spacer sequence comprising the nucleic acid sequence of SEQ ID NO: 15; and
p) a second AAV ITR sequence comprising the nucleic acid sequence of SEQ ID NO: 16.

11. The AAV piggyBac transposon polynucleotide of claims 8-10, wherein the AAV piggyBac transposon polynucleotide comprises the nucleic acid sequence of SEQ ID NO: 21.

12. A vector comprising the AAV piggyBac transposon polynucleotide of any claims 8-11.

13. The vector of claim 12, wherein the vector is an AAV viral vector.

14. The vector of claim 13, wherein the AAV viral vector is an AAV1, AAV2, AAV3, AAV4, AAV5, AAV6, AAV7, AAV8, AAV9, AAV10 or AAV11 viral vector.

15. The vector of claim 14, wherein the AAV viral vector is an AAV8 or AAV9 viral vector.

16. A pharmaceutical composition comprising the vector of claim 4-7 or 12-15.

17. A composition comprising: 1) the vector of claims 4-7 or 12-15; and 2) at least one LNP composition comprising at least one mRNA molecule encoding a transposase.

18. The composition of claim 17, wherein the mRNA molecule further comprises a 5′-CAP.

19. The composition of claim 17 or 18, wherein transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase.

20. The composition of any of claims 17-19, wherein the at least one LNP composition comprises:
about 54% of ssPalmO-Ph-P4C2 by moles,
about 35% of cholesterol by moles,
about 10% of DOPC by moles, and
about 1% of DMG-PEG2000 by moles.

21. A pharmaceutical composition comprising the composition of claims 17-20.

22. A method of treating phenylketonuria (PKU) in a subject in need thereof comprising administering to the subject at least one therapeutically effective dose of a composition comprising: a) the polynucleotide, vector or pharmaceutical composition of any one of the preceding claims; and b) at least one LNP composition comprising at least one mRNA molecule encoding a transposase.

23. The method of claim 22, wherein the at least one LNP composition comprises:
about 54% of ssPalmO-Ph-P4C2 by moles,
about 35% of cholesterol by moles,
about 10% of DOPC by moles, and
about 1% of DMG-PEG2000 by moles.

24. The method of claim 22 or 23, wherein the mRNA molecule further comprises a 5′-CAP.

25. The method of claims 22-24, wherein transposase is a piggyBac™ (PB) transposase, a piggyBac-like (PBL) transposase, a Super piggyBac™ (SPB) transposase, a Sleeping Beauty transposase, a Hyperactive Sleeping Beauty (SB100X) transposase, a helitron transposase, a Tol2 transposase, a TcBuster transposase or a mutant TcBuster transposase.

26. The method of claim 25, wherein the transposase is a Super piggyBac™ (SPB) transposase.
